(19) United States
(12) Patent Application Publication
(10) Pub. No.: US 2025/0177571 A1
(43) Pub. Date: Jun. 5, 2025

(54) SEQUENCE OF ACTIVATED PROTEIN C

(71) Applicant: JICHI MEDICAL UNIVERSITY, Tokyo (JP)

(72) Inventors: Tsukasa OHMORI, Shimotsuke (JP); Tomoki TOGASHI, Shimotsuke (JP); Morisada HAYAKAWA, Shimotsuke (JP); Nobuhiko KAMOSHITA, Shimotsuke (JP); Yuji KASHIWAKURA, Shimotsuke (JP); Takafumi HIRAMOTO, Shimotsuke (JP); Nemekhbayar BAATARTSOGT, Shimotsuke (JP)

(73) Assignee: JICHI MEDICAL UNIVERSITY, Tokyo (JP)

(21) Appl. No.: 18/844,794

(22) PCT Filed: Mar. 8, 2023
(86) PCT No.: PCT/JP2023/008880

§ 371 Date: Sep. 6, 2024

(30) Foreign Application Priority Data
Mar. 8, 2022 (JP) 2022-035659

(51) Int. Cl. A61K 48/00; A61K 38/48; C12N 9/64; C12N 15/86

(52) U.S. Cl. CPC A61K 48/0058; A61K 38/4866; C12N 9/6464; C12N 15/86; C12Y 304/21069; C12N 2750/14143; C12N 2830/008

(57) ABSTRACT

The invention provides a polypeptide or a partial polypeptide thereof, containing an amino acid sequence represented by the formula:

A₁—A₂—A₃ (I)

wherein A₁ is an amino acid sequence comprising an amino acid sequence of a light chain of protein C or a homologue thereof, A₂ is an amino acid sequence constituting a self-cleaving site, and A₃ is an amino acid sequence comprising an amino acid sequence of a heavy chain of protein C or a homologue thereof, wherein a dimeric protein or partial protein thereof consisting of fragments on the N-terminal side and C-terminal side of the cleavage site of A₂, has protein C activity. The polypeptide or partial polypeptide thereof makes it possible to 1) produce activated protein C as a recombinant preparation, and 2) link same to effective gene therapy for protein C deficiency.

### TECHNICAL FIELD

The present invention relates to a modified protein C polypeptide, and more specifically relates to a novel modified protein C polypeptide into which a self-cleaving site has been inserted, a pharmaceutical composition containing an activated protein C protein generated from the polypeptide, and the like.

### BACKGROUND ART

Protein S (PS), protein C (PC) and antithrombin (AT) deficiencies are the three major congenital thrombophilia in Japanese people. All of these are autosomal dominant (dominant) genetic diseases, and heterozygous mutation carriers develop deep vein thrombosis, while homozygous and compound heterozygous carriers of severe types develop purpura fulminans in newborn babies. A decrease in the activity of these three factors is identified in 65% of Japanese adults who developed deep vein thrombosis, and gene mutations are identified in about half of them. The full extent of the perinatal and maternal-fetal area is not clear.

Protein C is a blood protein that acts as a physiological anticoagulant factor. It is activated by thrombin at the site of thrombus formation, and exerts an anticoagulant action by cleaving activated factor V and activated factor VIII; it functions as a “coagulation brake.” Congenital abnormalities in the protein C gene (PROC) lead to a tendency toward thrombosis due to disruption of the balance between coagulation and anticoagulation.

The number of patients with congenital protein C deficiency is about 1 to 2 per 1,000 people (heterozygote is 0.16%, homozygote is about 1 per 500,000 people). The blood protein C activity in heterozygote patients decreases to 30 to 50% of normal. They are often asymptomatic until adolescence, but develop deep vein thrombosis, pulmonary embolism, and the like due to infection, trauma, surgery, pregnancy, and the like.

The blood protein C activity in homozygote patients decreases to less than 5% of normal. In the neonatal period, they cause a severe hemorrhagic symptom called purpura fulminans. In the case of heterozygotes, similar to other congenital thrombophilia, deep vein thrombosis of the lower limbs, thrombophlebitis, and associated pulmonary embolism recur from a young age. Arterial thrombosis is rare. In the case of homozygotes and double heterozygotes, a special condition called purpura fulminans occurs in the neonatal period, with intracranial hemorrhage and infarction, widespread purpura and hemorrhagic necrosis in the thigh, lower leg, buttock, abdomen, and scrotum, and further, multiple organ failure due to microthrombus.

Anact C (registered trademark), an activated protein C (APC) preparation, is used to treat neonatal purpura fulminans. Anact C (registered trademark) is derived from plasma and requires plasma for production. Therefore, there is a risk of unknown infections. In addition, Anact C has an extremely short half-life, requires continuous administration, and has no evidence of long-term use. The drug has a short half-life and requires continuous administration, and therefore, anticoagulant therapy with warfarin and the like is used during the stable phase. However, anticoagulant therapy is difficult to control, and a tendency toward thrombosis is developed easily or, conversely, a tendency toward bleeding is developed due to the drug. Cerebral hemorrhage associated with anticoagulation can be fatal at times. In addition, it has been reported that the blood concentration of protein C is 70 nM and the concentration of the active form is 40 pM, with the blood concentration of the active form remaining at less than 0.1% of the total. Therefore, the arrival of a revolutionary drug that can cure congenital protein C deficiency has been awaited.

Patent Literature 1 describes human protein C derivatives, and describes that the derivatives have increased anticoagulant activity compared to wild-type protein C and retain the biological activity of wild-type human protein C, require less frequent administration and/or lower dosages than wild-type human protein C in the treatment of acute coronary syndromes, vascular occlusive disorders, hypercoagulable states, thrombotic disorders, and disease states susceptible to thrombosis, and in particular, that when the amino acid Ser at position 11 of wild-type human protein C is replaced with Gly and the amino acid Ser at position 12 is replaced with Asn, the derivatives exhibit four times higher anticoagulant activity at maximum compared to the wild type.

Patent Literature 2 describes a human protein C derivative, and describes that the derivative has enhanced anticoagulant activity, resistance to serpin inactivation, and enhanced sensitivity to thrombin activation compared to wild-type protein C, and retains the biological activity of wild-type human protein C, and that the derivative may require less frequent administration and/or lower dosages than wild-type human protein C in the treatment of acute coronary syndromes, vascular occlusive disorders, hypercoagulable states, thrombotic disorders, and disease states susceptible to thrombosis. However, it does not show specific anticoagulant activity compared to the wild type.

Patent Literature 3 describes a protein C derivative, but does not disclose specific activity compared to the wild type.

Patent Literature 4 describes protein C derivatives, and describes that these polypeptides retain the biological activity of wild-type human protein C and have a substantially longer half-life in human blood, that these polypeptides require fewer administrations and/or smaller doses in the treatment of vascular occlusive diseases, hypercoagulable states, thrombosis diseases, and disease states predisposing to thrombosis, compared to wild-type human protein C, and that in particular, when the amino acid at position 194 of the wild-type protein C sequence was substituted from Leu to Ser, the stability in vivo became about four times higher than that of the wild type.

Patent Literature 5 describes a transformed cell containing a nucleotide sequence encoding a protein that is cleavable by furin and exhibiting an Arg-(Lys/Arg)-Arg motif, and describes protein C as a protein option. Patent Literature 6 describes a composition containing a modified blood coagulation factor having a proteolytic cleavage site that is not generally present and engineered to enable intracellular cleavage and secretion of an active form, and describes protein C as the blood coagulation factor.

However, Patent Literatures 1 to 4 do not describe the insertion of a self-cleaving site into protein C. Patent Literatures 5 and 6 also do not disclose or suggest an amino acid sequence in which a self-cleaving site has been inserted at a specific position in the protein C polypeptide of the present invention.

### CITATION LIST

### Patent Literature

[Patent Literature 1]
Japanese Translation of PCT Application Publication No. 2003-514545
[Patent Literature 2]
Japanese Translation of PCT Application Publication No. 2003-521938
[Patent Literature 3]
Japanese Translation of PCT Application Publication No. 2003-521919
[Patent Literature 4]
Japanese Translation of PCT Application Publication No. 2002-542832
[Patent Literature 5]
WO 2016/025615 A1
[Patent Literature 6]
WO 2001/070763 A1

### SUMMARY OF INVENTION

### Technical Problem

An object of the present invention is to provide an innovative activated protein C capable of curing protein C deficiency, a pharmaceutical composition containing the same, and the like.

### Means for Solving the Problem

The present inventors have prepared the polypeptide of the present invention by inserting a self-cleaving site into the site of protein C that is cleaved by thrombin. They expressed the polypeptide in cultured cells and found that protein C activity and anticoagulant action were observed in the culture supernatant without artificial activation. They also expressed the polypeptide of the present invention in mice in vivo using a viral vector and found that protein C activity and anticoagulant action were increased in the blood, which resulted in the completion of the present invention.

Accordingly, the present invention provides the following.
[1]A polypeptide or a partial polypeptide thereof, comprising an amino acid sequence represented by formula:

A₁—A₂—A₃ (I)
wherein A₁ is an amino acid sequence comprising an amino acid sequence of a light chain of protein C or a homologue thereof, A₂ is an amino acid sequence constituting a self-cleaving site, and A₃ is an amino acid sequence comprising an amino acid sequence of a heavy chain of protein C or a homologue thereof, wherein a dimeric protein consisting of fragments on the N-terminal side and C-terminal side of the cleaving site of A₂, or a partial protein thereof, has protein C activity.
[2] The polypeptide of [1] or partial polypeptide thereof, which satisfies the following conditions:
(1) an amino acid sequence (formula:

A₁—A₃ (II)

) in which A₁ (N-terminal side) and A₃ (C-terminal side) are linked comprises the amino acid sequence of SEQ ID NO:2;
(2) an amino acid sequence in which A1 (N-terminal side) and A₃ (C-terminal side) are linked comprises an amino acid sequence in which 1 to 45 amino acids have been deleted, substituted, inserted, or added in the amino acid sequence represented by SEQ ID NO:2; or
(3) an amino acid sequence in which A₁(N-terminal side) and A3 (C-terminal side) are linked comprises an amino acid sequence having 90% or more identity to the amino acid sequence represented by SEQ ID NO:2.
[3] The polypeptide or partial polypeptide thereof of [1] or
[2], wherein A2 is selected from the group consisting of RKRRKR (SEQ ID NO: 3), KRRKR (SEQ ID NO: 4), RKR, KR, RHQR (SEQ ID NO: 5), RSKR (SEQ ID NO: 6), ATNFSLLKQAGDVEENPGP (P2A) (SEQ ID NO: 7), RKRRKRRKR (SEQ ID NO: 8), and RKRRKRRKRRKR (SEQ ID NO: 9).
[4] The polypeptide or partial polypeptide thereof of any of [1] to [3], wherein A₂ is RKRRKR (SEQ ID NO: 3) or KRRKR (SEQ ID NO: 4).
[5] The polypeptide or partial polypeptide thereof of any of [1] to [4), wherein the polypeptide comprises the amino acid sequence represented by SEQ ID NO: 13 or SEQ ID NO: 14. [6]A dimeric protein or a partial protein thereof consisting of fragments on the N-terminal side and C-terminal side of the A2 cleaving site of the polypeptide or partial polypeptide of any of [1] to [5], wherein the protein or partial protein thereof has protein C activity.
[7]A nucleic acid comprising a nucleotide sequence encoding the polypeptide or partial polypeptide thereof of any of [1] to [5].
[8]A vector comprising the nucleic acid of [7].
[9] The vector of [8], which is an expression vector.
[10] The vector of [8], which is a donor vector.
[11] The vector of any of [8] to [10], which is a plasmid vector.
[12] The vector of any of [8] to [10], which is a viral vector.
[13] The vector of any of [8] to [10] and 12, which is an adeno-associated virus (AAV) vector.
[14]A host cell comprising the vector of any of [8] to [13].
[15]A host cell population comprising the host cell of [14].
[16]A pharmaceutical composition comprising the polypeptide or partial polypeptide thereof of any of [1] to [5], the protein or partial protein thereof of [6], the nucleic acid of [7], the vector of any of [8] to [13], the host cell of [14], or the host cell population of [15].
[17]A pharmaceutical composition comprising the vector of any of [10] to [13] and a vector comprising a nucleic acid encoding a nucleic acid metabolic enzyme.
[18] The pharmaceutical composition of [17], wherein the nucleic acid metabolic enzyme is a CRISPR/Cas9 system nucleic acid metabolic enzyme, and the composition further comprises a vector comprising a nucleic acid encoding a guide RNA, or comprises a vector comprising a nucleic acid encoding a guide RNA together with a nucleic acid encoding a nucleic acid metabolic enzyme.
[19] The pharmaceutical composition of any of [16] to [18], which is for suppressing blood coagulation.
[20] The pharmaceutical composition of any of [16] to [18], which is for treating or preventing thrombosis.
[21] The pharmaceutical composition of [20], wherein the thrombosis is selected from the group consisting of venous thrombosis, disseminated intravascular coagulation, (neonatal) purpura fulminans, deep vein thrombosis pulmonary thromboembolism, and thrombosis associated with new coronavirus infection.
[22]A method for producing a protein C-expressing cell, comprising introducing the vector of any of [8] to [13] into a mammalian cell in vitro.
[23]A method for producing a recombinant preparation of protein C, comprising producing a protein C-expressing cell by the method of [22], and isolating, purifying, and formulating activated protein C from the cell.
[24]A method for treating or preventing thrombosis, comprising administering to a subject the vector of [12] or [13] or the pharmaceutical composition of any of [16] to [18].
[25] The method of [24], wherein the thrombosis is selected from the group consisting of venous thrombosis, disseminated intravascular coagulation, (neonatal) purpura fulminans, deep vein thrombosis, and acute pulmonary thromboembolism.

### Effects of the Invention

By using the polypeptide of the present invention, activated protein C can be obtained from the culture supernatant of cultured cells expressing the polypeptide. In addition, by expressing the polypeptide of the present invention in vivo using a viral vector, protein C activity and anticoagulant action in blood can be increased.

### BRIEF DESCRIPTION OF THE DRAWINGS

FIG. 1 shows the structure of protein C and the inserted self-cleavage sequence.

FIG. 2 shows protein C activity in HEK293 cell supernatant (without snake venom for activation) (mean±SEM, n=3-4).

FIG. 3 shows the effect of cell supernatant in inhibiting human plasma coagulation. The supernatant of plasmid-transfected HEK293 cells was added to standard human plasma, and the coagulation time APTT (A: mean±SEM, n=3) and PT (B: mean±SEM, n=3) were measured. A concentration-dependent extension of the coagulation time was observed only when the self-cleavage sequence 2RKR was used.

FIG. 4 shows human protein C (hPC) activity and the APTT extension effect when AAV vector was administered to wild-type mice. An AAV vector expressing 2RKR was administered to wild-type mice. Blood was collected over time and hPC activity (left figure: mean±SEM, n=4) and coagulation time (APTT) (right figure: mean±SEM, n=4-5) were measured. An increase in human PC activity and an extension of APTT were observed in mouse blood after vector administration. (left figure: 1.2×10¹²/mouse, 4×10¹¹/mouse; right figure: 1.2×10¹²/mouse, 4×10¹¹/mouse, 4×10¹⁰/mouse)

FIG. 5 shows a schematic diagram of the AAV8 vector (Cas9) expressing SaCas9 used for genome editing therapy, and the AAV8 vector (Donor) with a wild-type PC sequence bound to a P2A sequence with a homologous recombination sequence (about 1 kb) at the gene locus on both ends, with diagrams showing the site of the homologous recombination sequence on the genome and the double-strand cleaving site (upper figure), as well as a figure showing the PC activity measured in newborn mice administered with these vectors (lower figure: mean±SEM, n=4). [FIG. 6]

FIG. 6 shows the results of a test of the blood coagulation inhibitory effect of activated mouse protein C. AAV8 vectors expressing wild-type mouse protein C sequence (mPC) or modified mouse protein C sequence (mPC variant) were administered intravenously once, and blood was drawn 4 to 8 weeks after administration to obtain plasma. Increase in protein C antigen level in plasma (A: mean±SEM, n=4), coagulation time [activated partial thromboplastin time (APTT)](B: mean±SEM, n=4), factor V activity (C: mean±SEM, n=4), and factor VIII activity (D: mean±SEM, n=4 (mPC Medium group: n=2, mPC-2RKR High group: n=3)) were measured. Only the modified protein C sequence showed vector dose-dependent inhibition of coagulation.

FIG. 7 shows the measurement results of the amount of AAV genome in the liver of mice that received a single intravenous administration of an AAV8 vector expressing a wild-type mouse protein C sequence (mPC) or a modified mouse protein C sequence (mPC variant) (mean±SEM, n=4). The amount of AAV genome in the liver did not change in any group.

FIG. 8 shows the confirmation results of reactive oxygen-dependent pathological thrombus formation in the scavenger vein of mice that received a single intravenous administration of an AAV8 vector expressing a wild-type mouse protein C sequence (mPC) or a modified mouse protein C sequence (mPC variant) (left figure: tissue staining, right figure: quantification of angiogenesis per vessel area, mean±SEM, n=3-4, *P<0.05, two-tailed Student's t-test). Pathological thrombus formation was suppressed with the mPC variant.

FIG. 9 shows the evaluation results of the phenotype of protein C-deficient mice. Two types of AAV vectors were administered to neonate mice (Proc−/−) born from the mating of heterozygous protein C-deficient mice (Proc+/−), and activated protein C was expressed in the neonate liver by genome editing. Similar to the mating of hemophilia A mice (F8−/−), expression of activated protein C by genome editing (Treated) resulted in the survival of protein C-deficient mice (A). Mouse protein C antigen amount (B), factor V activity (C), factor VIII activity (D), and coagulation time [activated partial thromboplastin time (APTT)](E) were measured. Genome editing treatment to express modified protein C enabled protein C-deficient mice to survive.

### DESCRIPTION OF EMBODIMENTS

### 1. Polypeptide

The present invention provides a polypeptide or a partial polypeptide thereof, comprising an amino acid sequence represented by formula: A₁—A₂—A₃ (I)
wherein A₁ is an amino acid sequence comprising an amino acid sequence of a light chain of protein C or a homologue thereof, A₂ is an amino acid sequence constituting a self-cleaving site, and A₃ is an amino acid sequence comprising an amino acid sequence of a heavy chain of protein C or a homologue thereof, wherein a dimeric protein consisting of fragments on the N-terminal side and C-terminal side of the cleaving site of A₂, or a partial protein thereof, has protein C activity.

In the present specification, amino acid residues and peptides are written with the N-terminus on the left and the C-terminus on the right, according to the standard method, unless otherwise specified.

In the present specification, the term “protein C” refers to a precursor of vitamin K-dependent serine protease, which is one of the blood coagulation control regulators in the blood coagulation system of vertebrates, is activated by a complex of thrombin generated during the blood coagulation process and thrombomodulin on vascular endothelial cells, and specifically inactivates blood coagulation factors Vₐ and VIIIₐ through proteolysis. Most protein C is a heterodimer consisting of a heavy chain and a light chain, modified with sugar chains, and linked by a disulfide bond between a cysteine residue in the heavy chain and a cysteine residue in the light chain. In human protein C, the heavy chain (41 kDa) and light chain (21 kDa) are linked by a disulfide bond between Cys183 and Cys319.

Protein C is mainly synthesized in the liver. For example, human protein C is translated as a polypeptide of 461 amino acids in full length, including, from the N-terminus side, a signal peptide (positions 1-32), a light chain (positions 43-88) (the light chain includes a G1a domain (positions 43-88), a helical aromatic segment (positions 89-96), and two EGF-like domains (positions 97-132, 136-176)), an activation peptide (positions 200-211), and a heavy chain (positions 212-461) (the heavy chain includes a trypsin-like serine protease domain (positions 212-450)).

In the present specification, the term “activated protein C” refers to a form having serine protease activity that is generated when the activation peptide bound to the N-terminus of the heavy chain of protein C is cleaved by thrombin.

In the present specification, the term “activated protein C” refers to a dimeric protein consisting of fragments on the N-terminal side and C-terminal side which are formed by self-cleaving of A₂ from the polypeptide of the present invention, or a partial protein thereof.

In the present specification, the term “protein C activity” refers to a serine protease activity, and to the activity of specifically inactivating blood coagulation factors Vₐ and VIIIₐ through proteolysis in the blood coagulation system of a species of organism of the same origin as the protein C. Protein C activity can be measured, in the case of human protein C activity in plasma, for example, using the activity measuring reagent, Verichrome Protein C (Sysmex, Kobe, Japan) with a fully automated blood coagulation measuring device CS1600 (Sysmex). The kit consists of the following:
1. Protein C activator
Snake venom 3.0 U/vial
2. Substrate reagent
Pyro-glutamic acid-proline0arginine-methoxynitroanilide (p-Glu-Pro-Arg-MNA) 4 mmol/L
3. Buffer solution

In the present specification, the origin of protein C is not particularly limited as long as it is a vertebrate, and examples of the vertebrate include mammals, birds, reptiles, amphibians, fish, and the like. Examples of the fish include tilapia, sea bream, flounder, shark, and salmon; examples of the amphibians include frogs and newts; examples of the reptiles include crocodiles, turtles, and lizards; examples of the birds include chickens, quails, ducks, geese, ostriches, and guinea fowl; and examples of the mammals include rodents such as mice, rats, hamsters, and guinea pigs, laboratory animals such as rabbits, livestock such as pigs, cows, goats, horses, sheep, and minks, pets such as dogs and cats, humans, monkeys, cynomolgus monkeys, rhesus monkeys, marmosets, orangutans, and chimpanzees. Such mammals are preferred, and among them, humans are particularly preferred.

In the present specification, a “homolog” of protein C refers to either or both of the following (A) and (B): (A) A molecule consisting of an amino acid sequence of protein C in which 1 to X amino acids have been deleted, substituted, inserted, or added. Here, X is 10% or less, 9% or less, 8% or less, 7% or less, 6% or less, 5% or less, 4% or less, 3% or less, 2% or less, 1% or less, or 0.5% or less of the number of amino acids in the full-length precursor of protein C. (B) A molecule consisting of an amino acid sequence having an identity of 90% or more, 91% or more, 92% or more, 93% or more, 94% or more, 95% or more, 96% or more, 97% or more, 98% or more, 99% or more, 99.1% or more, 99.2% or more, 99.3% or more, 99.4% or more, 99.5% or more, 99.6% or more, 99.7% or more, 99.8% or more, or 99.9% or more and less than 100% with a protein C amino acid sequence.

Here, “identity” refers to the proportion (%) of identical and similar amino acid residues to the total overlapping amino acid residues in optimal alignment when two amino acid sequences are aligned using a mathematical algorithm known in the art (preferably, the algorithm can take into account the introduction of gaps into one or both of the sequences for optimal alignment). “Similar amino acids” refer to amino acids that are similar in physicochemical properties, and examples of such amino acids include aromatic amino acids (Phe, Trp, Tyr), aliphatic amino acids (Ala, Leu, Ile, Val), polar amino acids (Gln, Asn), basic amino acids (Lys, Arg, His), acidic amino acids (Glu, Asp), amino acids with hydroxyl groups (Ser, Thr), and amino acids with small side chains (Gly, Ala, Ser, Thr, Met) that are classified into the same group. It is predicted that substitution with such similar amino acids will not cause a change in the phenotype of the protein (i.e., conservative amino acid substitution). Specific examples of conservative amino acid substitution are well known in the art and are described in various documents (see, for example, Bowie et al., Science, 247:1306-1310 (1990)). The identity of amino acid sequences in the present specification can be calculated using the homology calculation algorithm NCBI BLAST (National Center for Biotechnology Information Basic Local Alignment Search Tool) under the following conditions (expectation value=10; gaps allowed; matrix=BLOSUM62; filtering=OFF).

In one embodiment, the polypeptide of the present invention satisfies the following conditions:
(1) an amino acid sequence (formula: A₁-A₃ (II)) in which A₁ (N-terminal side) and A₃ (C-terminal side) are linked 5 comprises the amino acid sequence of SEQ ID NO:2;
(2) an amino acid sequence in which A₁ (N-terminal side) and A₃ (C-terminal side) are linked comprises an amino acid sequence in which 1 to 45 amino acids have been deleted, substituted, inserted, or added in the amino acid sequence represented by SEQ ID NO:2; or
(3) an amino acid sequence in which A₁ (N-terminal side) and A₃ (C-terminal side) are linked comprises an amino acid sequence having 90% or more identity to the amino acid sequence represented by SEQ ID NO:2.

In one embodiment, the amino acid sequence in which A₁ (N-terminal side) and A₃ (C-terminal side) are linked is an amino acid sequence in which 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 1-26, 1-27, 1-28, 1-29, 1-30, 1-31, 1-32, 1-33, 1-34, 1-35, 1-36, 1-37, 1-38, 1-39, 1-40, 1-41, 1-42, 1-43, 1-44, 1-45, 1-46, 1-47, 1-48, 1-49, 1-50 amino acids have been deleted, substituted, inserted, or added in the amino acid sequence represented by SEQ ID NO:2.

Preferably, the amino acid sequence in which A₁ (N-terminal side) and A₃ (C-terminal side) are linked is an amino acid sequence in which 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 1-26, 1-27, 1-28, 1-29, 1-30, 1-31, 1-32, 1-33, 1-34, 1-35, 1-36, 1-37, 1-38, 1-39, 1-40, 1-41, 1-42, 1-43, 1-44, 1-45 amino acids have been deleted, substituted, inserted, or added in the amino acid sequence represented by SEQ ID NO:2.

In one embodiment, the amino acid sequence in which A₁ (N-terminal side) and A₃ (C-terminal side) are linked has 70% or more, 75% or more, 80% or more, 85% or more, 90% or more, 91% or more, 92% or more, 93% or more, 94% or more, 95% or more, 96% or more, 97% or more, 98% or more, 99% or more, 99.1% or more, 99.2% or more, 99.3% or more, 99.4% or more, 99.5% or more, 99.6% or more, 99.7% or more, 99.8% or more, or 99.9% or more identity, or 100% identity, to the amino acid sequence represented by SEQ ID NO:2.

In one embodiment, the polypeptide of the present invention is a polypeptide in which
(a-1) A₁ contains an amino acid sequence represented by SEQ ID NO:15;
(a-2) A₁ contains an amino acid sequence in which 1 to 20 amino acids have been deleted, substituted, inserted, or added in the amino acid sequence represented by SEQ ID NO:15; or
(a-3) A₁ contains an amino acid sequence having 90% or more identity to the amino acid sequence represented by SEQ ID NO:15; and
(b-1) A₃ contains an amino acid sequence represented by SEQ ID NO: 16;
(b-2) A₃ contains an amino acid sequence in which 1 to 30 amino acids have been deleted, substituted, inserted, or added in the amino acid sequence represented by SEQ ID NO:16; or
(b-3) A₃ contains an amino acid sequence having 90% or more identity to the amino acid sequence represented by SEQ ID NO:16, and the activated protein formed by self-cleaving of A₂ has protein C activity.

In one embodiment, the polypeptide of the present invention is a polypeptide in which
(c-1) A₁ contains the amino acid sequence represented by SEQ ID NO:15, and A₃ contains the amino acid sequence represented by SEQ ID NO:16;
(c-2) A₁ contains the amino acid sequence in which 1 to 50 amino acids have been deleted, substituted, inserted, or added in the amino acid sequence represented by SEQ ID NO:15, and A₃ contains the amino acid sequence in which 1 to 50 amino acids have been deleted, substituted, inserted, or added in the amino acid sequence represented by SEQ ID NO:16; or
(c-3) A₁ contains an amino acid sequence having 90% or more identity to the amino acid sequence represented by SEQ ID NO:15, and A₃ contains an amino acid sequence having 90% or more identity to the amino acid sequence represented by SEQ ID NO:16, and the activated protein formed by self-cleaving of A₂ has protein C activity.

In one embodiment, the amino acid sequence contained in A₁ is an amino acid sequence in which 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20 amino acids have been deleted, substituted, inserted, or added in the amino acid sequence represented by SEQ ID NO:15.

Preferably, the amino acid sequence contained in A₁ is an amino acid sequence in which 1-2, 1-3, 1-4, 1-5, 1-6, 1-7, 1-8, 1-9, 1-10, 1-11, 1-12, 1-13, 1-14, 1-15, 1-16, 1-17 amino acids have been deleted, substituted, inserted, or added in the amino acid sequence represented by SEQ ID NO:15.

In one embodiment, the amino acid sequence contained in A₃ is an amino acid sequence in which 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 1-26, 1-27, 1-28, 1-29, 1-30 amino acids have been deleted, substituted, inserted, or added in the amino acid sequence represented by SEQ ID NO:16.

Preferably, the amino acid sequence contained in A₃ is an amino acid sequence in which 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25 amino acids have been deleted, substituted, inserted, or added in the amino acid sequence represented by SEQ ID NO:16.

In one embodiment, the amino acid sequence contained by A₁ has 70% or more, 75% or more, 80% or more, 85% or more, 90% or more, 91% or more, 92% or more, 93% or more, 94% or more, 95% or more, 96% or more, 97% or more, 98% or more, 99% or more, 99.1% or more, 99.2% or more, 99.3% or more, 99.4% or more, 99.5% or more, 99.6% or more, 99.7% or more, 99.8% or more, or 99.9% or more identity, or 100% identity, to the amino acid sequence represented by SEQ ID NO:15.

Preferably, the amino acid sequence contained in A₁ has 90% or more, 91% or more, 92% or more, 93% or more, 94% or more, 95% or more, 96% or more, 97% or more, 98% or more, 99% or more, 99.1% or more, 99.2% or more, 99.3% or more, 99.4% or more, 99.5% or more, 99.6% or more, 99.7% or more, 99.8% or more, or 99.9% or more identity, or 100% identity, to the amino acid sequence represented by SEQ ID NO:15.

In one embodiment, the amino acid sequence contained in A₃ has 70% or more, 75% or more, 80% or more, 85% or more, 90% or more, 91% or more, 92% or more, 93% or more, 94% or more, 95% or more, 96% or more, 97% or more, 98% or more, 99% or more, 99.1% or more, 99.2% or more, 99.3% or more, 99.4% or more, 99.5% or more, 99.6% or more, 99.7% or more, 99.8% or more, or 99.9% or more identity, or 100% identity, to the amino acid sequence represented by SEQ ID NO:16.

Preferably, the amino acid sequence contained in A₃ has 90% or more, 91% or more, 92% or more, 93% or more, 94% or more, 95% or more, 96% or more, 97% or more, 98% or more, 99% or more, 99.1% or more, 99.2% or more, 99.3% or more, 99.4% or more, 99.5% or more, 99.6% or more, 99.7% or more, 99.8% or more, or 99.9% or more identity, or 100% identity, to the amino acid sequence represented by SEQ ID NO:16.

In the present specification, the amino acid sequence represented by SEQ ID NO:15 contains the sequence of the light chain of human wild-type protein C, and the amino acid sequence represented by SEQ ID NO:16 contains the sequence of the heavy chain of human wild-type protein C.

In the present specification, the term “partial polypeptide” refers to a part of a polypeptide that contains a consecutive amino acid sequence of the polypeptide. In addition, when referring to a “partial polypeptide” of the polypeptide of the present invention, the “partial polypeptide” includes a part of the consecutive amino acid sequence of the polypeptide of the present invention, and the entire sequence of A₂.

In the present specification, the term “amino acid sequence constituting a self-cleaving site” refers to an amino acid sequence of 50 amino acid length or less, 45 amino acid length or less, 40 amino acid length or less, 35 amino acid length or less, 30 amino acid length or less, 25 amino acid length or less, 20 amino acid length or less, 15 amino acid length or less, 14 amino acid length or less, 13 amino acid length or less, 12 amino acid length or less, 11 amino acid length or less, 10 amino acid length or less, 9 amino acid length or less, 8 amino acid length or less, 7 amino acid length or less, 6 amino acid length or less, or 5 amino acid length or less that includes a site where self-cleaving of the peptide bond in the main chain occurs in a polypeptide or peptide.

In the present specification, “self-cleaving” refers to cleaving due to the autolytic activity of a polypeptide or peptide consisting of an amino acid sequence constituting the self-cleaving site, as well as cleaving of the cleavage site by another molecule (e.g., protease) present in a system for producing the polypeptide or partial polypeptide thereof of the present invention, or the protein or partial protein thereof of the present invention. In one embodiment, the self-cleaving site contains an amino acid sequence that is specifically recognized by another molecule (e.g., protease) present in a system for producing the protein or partial protein thereof of the present invention. It also refers to cleaving caused by ribosome skipping of a peptide bond between amino acids in the amino acid sequence constituting the self-cleaving site during polypeptide synthesis.

In the polypeptide of the present invention, A₂ may be, for example, RKRRKR (SEQ ID NO: 3), KRRKR (SEQ ID NO: 4), RKR, KR, RHQR (SEQ ID NO: 5), RSKR (SEQ ID NO: 6), ATNFSLLKQAGDVEENPGP (P2A) (SEQ ID NO: 7), RKRRKRRKR (SEQ ID NO: 8), RKRRKRRKRRKR (SEQ ID NO: 9), EGRGSLLTCGDVEENPGP (T2A) (SEQ ID NO: 10), QCTNYALLKLAGDVESNPGP (E2A) (SEQ ID NO: 11), VKQTLNFDLLKLAGDVESNPGP (F2A) (SEQ ID NO: 12), preferably RKRRKR (SEQ ID NO: 3), KRRKR (SEQ ID NO: 4), RKR, KR, RHQR (SEQ ID NO: 5), RSKR (SEQ ID NO: 6), ATNFSLLKQAGDVEENPGP (P2A) (SEQ ID NO: 7), further preferably RKRRKR (SEQ ID NO: 3) and KRRKR (SEQ ID NO: 4).

In one embodiment, the polypeptide of the present invention contains the amino acid sequence represented by SEQ ID NO: 13 or SEQ ID NO: 14, or consists of the amino acid sequence represented by SEQ ID NO: 13 or SEQ ID NO: 14.

### 2. Protein

The polypeptide of the present invention becomes activated protein C when the amino acid sequence constituting the self-cleaving site is cleaved at the cleavage site (hereinafter also to be referred to as the protein of the present invention).

In the present specification, the term “partial protein” refers to a part of the protein, including a part of the continuous amino acid sequence of the protein. The “partial protein” of the protein of the present invention includes the entire fragment on the N-terminal side and a part of fragment on the C-terminal side of the A₂ cleavage site of the polypeptide of the present invention, a part of fragment on the N-terminal side and the entire fragment on the C-terminal side, or a part of fragment on the N-terminal side and a part of 25 fragment on the C-terminal side. In one embodiment, the “partial protein” of the protein of the present invention is generated by processing the protein of the present invention in vivo and in cells. In one embodiment, in the “partial protein” of the present invention, the amino acid sequence of a fragment on the N-terminal side of the A₂ cleavage site of the polypeptide of the present invention is the same as the amino acid sequence of the light chain of wild-type protein C.

In one embodiment, the polypeptide or partial polypeptide thereof, and the protein or partial protein thereof of the present invention are preferably isolated. “Isolated” means that an operation for removing factors other than the target components has been performed, and they are no longer in a naturally occurring state. The purity of the “isolated protein or partial protein thereof” (the percentage by weight of the target protein or partial protein thereof in the total weight of the object to be evaluated) is generally 70% or more, preferably 80% or more, more preferably 90% or more, and further preferably 99% or more.

The polypeptide or partial polypeptide thereof, and protein or partial protein thereof of the present invention may be in the form of a salt. For example, a salt with a physiologically acceptable acid (e.g., inorganic acid, organic acid) or a base (e.g., alkali metal) is used, and a physiologically acceptable acid addition salt is particularly preferred. Examples of such salts include salts with inorganic acids (e.g., hydrochloric acid, phosphoric acid, hydrobromic acid, sulfuric acid) and salts with organic acids (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid).

### 3. Nucleic Acid

The present invention provides a nucleic acid containing a nucleotide sequence encoding the polypeptide or partial polypeptide of the present invention.

The nucleic acid encoding the polypeptide or partial polypeptide of the present invention may be DNA or RNA, or may be a DNA/RNA chimera. The nucleic acid may be double-stranded or single-stranded. In the case of a double strand, it may be double strand DNA, double strand RNA, or a DNA:RNA hybrid. In the case of a single strand, it may be a sense strand (i.e., coding strand) or an antisense strand (i.e., non-coding strand).

The nucleic acid of DNA encoding the polypeptide or partial polypeptide thereof of the present invention may be synthetic DNA or the like. For example, it may be obtained by amplifying full length Protein C cDNA (for example, the base sequence represented by SEQ ID NO: 1 in the case of humans) by Reverse Transcriptase-PCR (hereinafter abbreviated as “RT-PCR”) using total RNA or an mRNA fraction prepared from cells or tissues as a template. Alternatively, it may also be obtained by cloning from a cDNA library prepared by inserting the above-mentioned cDNA (fragment) into an appropriate vector, by colony or plaque hybridization method, PCR method, or the like. The vector used for the library may be any such as bacteriophage, plasmid, cosmid, phagemid, and the like.

The nucleic acid of the present invention may be functionally linked to an enhancer, promoter, transcription initiation signal, splicing signal, transcription termination signal, polyA addition signal, cap structure, 5′ untranslated region, Kozak sequence, internal ribosome entry site (IRES), 3′ untranslated region, and the like, which can exert activity in a host cell. By having such a configuration, the nucleic acid of the present invention is more stably transcribed and translated in a host cell. In addition, the nucleic acid of the present invention may be linked to a sequence (homology arm) homologous to a sequence before and after a site in the genome sequence of an organism. By having such a configuration, the nucleic acid of the present invention can be incorporated into the genome sequence of an organism by homologous recombination.

In one embodiment of the present invention, the nucleic acid contains a codon-optimized nucleotide sequence that encodes the polypeptide or partial polypeptide thereof described in the present specification. It is considered that optimization of a codon of a nucleotide sequence increases the translation efficiency of mRNA transcription products. Optimization of the codon of a nucleotide sequence can include replacing a native codon with another codon that codes for the same amino acid but can be translated by a tRNA that can be more easily utilized in the cell, thus increasing translation efficiency. Optimization of nucleotide sequence can also reduce secondary structure in the mRNA that interferes with translation, thus increasing translation efficiency.

The present invention also provides a nucleic acid containing a nucleotide sequence that is complementary to any of the nucleotide sequences of the nucleic acids described in the present specification or that hybridizes under stringent conditions to any of the nucleotide sequences of the nucleic acids described in the present specification.

The nucleotide sequence that hybridizes under stringent conditions preferably hybridizes under high stringency conditions. “High stringency conditions” means that a nucleotide sequence specifically hybridizes to a nucleotide sequence of a nucleic acid described in the present specification, in an amount detectably higher than nonspecific hybridization. High stringency conditions include low salt 25 conditions and/or high temperature conditions, such as, for example, a temperature of about 50-70° C., about 0.02-0.1 M NaCl, and the like. It is generally understood that the conditions can be made more stringent by increasing the amount of formamide to be added.

The present invention also provides a nucleic acid containing a nucleotide sequence that is at least about 70% or more, for example, about 80%, about 90%, about 91%, about 92%, about 93%, about 94%, about 95%, about 96%, about 97%, about 98% or about 99%, identical to the nucleic acid described in the present specification (nucleic acid containing the sequence of SEQ ID NO: 1, 17, or 18). Relating thereto, the nucleic acid may consist of the nucleotide sequence described in the present specification.

### 4. Vector

The present invention provides a vector containing the nucleic acid of the present invention. The vector may be a cloning vector, and may be a vector such as bacteriophage, plasmid, cosmid, or phagemid.

### (Expression Vector)

In one embodiment, the vector is an expression vector. In the expression vector, the above-mentioned nucleic acid of the present invention or a nucleic acid encoding the same is functionally linked to a promoter capable of exerting promoter activity in the cells of an organism to be the administration target (also called host cells, for example, human cells).

Those skilled in the art can select an appropriate promoter according to the type of host cell.

For example, when the host cell is an Escherichia bacterium, the trp promoter, lac promoter, recA promoter, APL promoter, lpp promoter, T7 promoter, and the like are used, but the promoters are not limited to these.

When the host cell is a Bacillus bacterium, the SPO1 promoter, SPO2 promoter, penP promoter, and the like are used, but the promoters are not limited to these.

When the host cell is a yeast cell, the Gall promoter, Gall/10 promoter, PHO5 promoter, PGK promoter, GAP promoter, ADH promoter, and the like are used, but the promoters are not limited to these.

When the host cell is an insect cell, the polyhedrin promoter, P10 promoter, and the like are used, but the promoters are not limited to these.

When the host cell is a plant cell, the CaMV35S promoter, CaMV19S promoter, NOS promoter, and the like are used, but the promoters are not limited to these.

When the host cell is a vertebrate cell, the SRa promoter, SV40 promoter, LTR promoter, CMV (cytomegalovirus) promoter, RSV (Rous sarcoma virus) promoter, MoMuLV (moloney murine leukemia virus) LTR, HSV-TK (herpes simplex virus thymidine kinase) promoter, and the like are used, but the promoters are not limited to these.

In particular, when the host cell is a human cell, pol I promoters, pol II promoters, pol III promoters, and the like can be used. Specifically, viral promoters such as SV40-derived early promoter, cytomegalovirus LTR, mammalian constitutive protein gene promoters such as the P-actin gene promoter, and RNA promoters such as tRNA promoters, and the like are used. When RNA expression is intended, it is preferable to use a pol III promoter as the promoter. Examples of pol III promoters include U6 promoter, H1 promoter, and tRNA promoter.

In addition to the above, the expression vector may contain an enhancer, a splicing signal, a polyA addition signal, a selection marker, an SV40 origin of replication (hereinafter sometimes abbreviated as SV40 ori), and the like, when desired. Examples of selection markers include the dihydrofolate reductase gene (hereinafter sometimes abbreviated as dhfr, methotrexate (MTX) resistance), the neomycin resistance gene (hereinafter sometimes abbreviated as neor, G418 resistance), and the like. In particular, when dhfr gene-deficient Chinese hamster cells are used and the dhfr gene is used as a selection marker, the target gene can also be selected by a thymidine-free medium.

The expression vector of the present invention may be a vector selected from the group consisting of the pUC series (Fermentas Life Sciences), pBluescript series (Stratagene, LaJolla, CA), pET series (Novagen, Madison, WI), pGEX series (Pharmacia Biotech, Uppsala, Sweden), and pEX series (Clontech, Palo Alto, CA). Bacteriophage vectors such as λGT10, AGT11, λZapII (Stratagene), AEMBL4, and ANM1149 may also be used. Examples of plant expression vectors include pBI01, pBI101.2, pBI101.3, pBIl21, and pBIN19 (Clontech). Examples of animal expression vectors include pEUK-C1, pMAM, and pMAMneo (Clontech). Preferably, the recombinant expression vector is a viral vector (e.g., AAV vector). In a preferred embodiment, the recombinant expression vector is an AAV vector carrying an ITR.

### (Donor Vector)

The present invention provides a donor vector capable of providing the above-mentioned nucleic acid of the present invention as a donor DNA in genetic homologous recombination. In one embodiment, the donor vector contains a sequence homologous to the sequence before and after the site in the 20 genomic sequence of the cell of the organism to be the administration target (also called a host cell, for example, a human cell), into which the above-mentioned nucleic acid of the present invention is incorporated by homologous recombination. In one embodiment, the site in the genome sequence is not 25 particularly limited, and is preferably a sequence that is inserted between genes and does not affect survival of the cell even when a change occurs in the sequence. In another embodiment, in the donor vector, the nucleic acid of the present invention is linked to a sequence (homology arm) that is homologous to the sequence before and after the site in the genome sequence of the cell of the organism to be the administration target, into which the nucleic acid of the present invention is incorporated by homologous recombination. In one embodiment, the site in the genome sequence may be a site present in a gene on the genome, and an example of such gene is a site in the Alb (ALB) locus.

In one embodiment, the homologous sequence includes a selected target nucleotide sequence (described below) in the sequence, and has a sufficient degree of sequence identity and length to cause homologous recombination with respect to the genomic DNA containing the sequence when DNA (donor DNA (e.g., donor vector) and/or genomic double-strand DNA) is cleaved within the target nucleotide sequence. In one embodiment, the homologous sequence includes a sequence homologous to (a part of) a sequence upstream and/or downstream of the selected target nucleotide sequence in the sequence, is linked to the nucleic acid of the present invention as a homology arm, and has a sufficient degree of sequence identity and length to cause homologous recombination with respect to the genomic DNA containing the sequence when genomic double-strand DNA is cleaved within the target nucleotide sequence.

The degree of identity of the homologous sequence to the sequence is not particularly limited as long as it allows homologous recombination. The degree of identity that allows homologous recombination varies depending on the length of the polynucleotide. For example, it may be at least about 80% or more, preferably at least about 85% or more, more preferably at least about 90% or more, and most preferably about 95-100%.

The length of the homologous sequence of the sequence is not particularly limited as long as it is long enough for homologous recombination with genomic DNA to occur. However, generally speaking, the longer the homologous region, the more efficiently the homologous recombination with genomic DNA occurs. On the other hand, the length of DNA that can be inserted is limited to a certain level by the efficiency of introduction of donor DNA (e.g., donor vector) into cells. Therefore, taking these into consideration, the length of the homologous sequence may be, for example, 0.15 kb to 20 kb, 0.18 kb to 10 kb, 0.2 kb to 8 kb, 0.3 kb to 5 kb, 0.5 kb to 2 kb, or 0.7 kb to 1 kb.

In one embodiment, the homologous sequence may be a nucleotide sequence that is homologous to the sequence or a partial sequence thereof present in the genome.

The homologous sequence can be cloned, for example, by synthesizing an oligo DNA primer based on the DNA sequence information of the sequence so as to cover a region that codes for a desired portion (a portion including a target nucleotide sequence described below), and amplifying the sequence by PCR using genomic DNA prepared from a host cell as a template. The sequence may also be cloned from a species other than the host cell, as long as the degree of identity that enables the above-mentioned homologous recombination is maintained.

The donor vector may further contain a selection marker gene for selecting a transformant in which the nucleic acid of the present invention has been inserted into the genome. Examples of the selection marker gene include, but are not limited to, genes that confer resistance to drugs such as tetracycline, ampicillin, and kanamycin, and genes that complement auxotrophic mutations. The gene that complements auxotrophic mutations is used in combination with a host cell having the corresponding auxotrophic mutation.

The donor vector used in the method of the present invention may be a double strand DNA, a single strand DNA (circular double strand DNA, linear double strand DNA, circular single strand DNA, linear single strand DNA), or a circular double strand DNA containing a single strand DNA. When the donor vector is a single strand DNA, “bp” is to be read as “b”.

While the donor vector is described mainly using a circular double strand DNA as a representative example, it will be readily understood by those skilled in the art that the description is similarly applicable to other donor DNAs other than circular double strand DNA.

In one embodiment, the circular double strand DNA is a circular double strand DNA plasmid. Examples of circular double strand DNA plasmids include, but are not limited to, plasmids derived from E. coli (e.g., pBR322, pBR325, pUC12, pUC13); plasmids derived from Bacillus subtilis (e.g., pUB110, pTP5, pC194); yeast-derived plasmids (e.g., YCplac33, pRS403, YIplac128); insect cell expression plasmids (e.g., pFast-Bac); plant cell expression plasmids (e.g., pBI01, pBI101.2, pBI101.3, pBIl21, and pBIN19); animal cell expression plasmids (e.g., pCAGGS, pSRa, pA1-11, pXT1, pRc/CMV, pRc/RSV, pcDNAI/Neo).

In one embodiment, the donor vector of the present invention may be an adeno-associated virus (AAV) vector, an adenovirus vector, a lentivirus vector, a Sendai virus vector, or a retrovirus vector. In a preferred embodiment, the vector is an AAV vector. The AAV vector may be a hepatocyte-tropic serotype AAV2, AAV3, AAV6, AAV7, AAV8, or AAV9 vector.

Adeno-associated viruses are members of the parvoviridae family and contain a linear, single strand DNA genome of less than about 5 kb nucleotides. AAV requires co-infection with a helper virus (i.e., adenovirus or herpesvirus) or expression of a helper gene for efficient replication. Typically, AAV vectors used for administration of therapeutic nucleic acids have about 96% of the parent genome deleted, leaving only the inverted terminal repeats (ITRs) containing the recognition signals for DNA replication and packaging. This eliminates immunological or toxic side effects due to viral gene expression. Furthermore, delivery of specific AAV proteins to the cells to be produced enables integration of AAV vectors containing AAV ITRs into specific regions of the genome of the cells when needed (see, for example, U.S. Pat. Nos. 6,342,390 and 6,821,511). Host cells containing integrated AAV genomes do not show changes in cell growth or morphology (see, for example, U.S. Pat. No. 4,797,368).

### 5. Host Cell and Cell Population

The present invention provides a host cell containing the vector of the present invention. As used in the present specification, the term “host cell” refers to any type of cell that can contain the vector of the present invention. The host cell may be a eukaryotic cell, e.g., a plant, an animal, a fungus, or an alga, or a prokaryotic cell, e.g., a bacterium. The host cell may be a cultured cell or a primary cell, i.e., directly isolated from an organism, e.g., human. The host cell may be an adherent cell or a suspension cell. Suitable host cells are known in the art, e.g., DH5a E. coli cells, Chinese hamster ovary cells, monkey VERO cells, COS cells, HEK293 cells, and the like. In the case of amplifying or replicating a vector, the host cell may be, for example, a prokaryotic cell, e.g., DH5a cells. In the case of producing a polypeptide or portion or a protein or portion of the present invention, the host cell may be, for example, a vertebrate cell. Preferably, the host cell is a mammalian cell, more preferably a human cell.

The type, tissue of origin, and developmental stage of the host cell are not limited. In one embodiment, the host cell is not a transformed cell containing a nucleotide sequence encoding a proteolytic enzyme (e.g., furin).

### 6. Pharmaceutical Composition

The present invention provides pharmaceutical compositions containing the polypeptide or partial polypeptide thereof, protein or partial protein thereof, nucleic acid, vector, host cell, or host cell population of the present invention.

The pharmaceutical composition of the present invention can contain any carrier, for example, a pharmaceutically acceptable carrier, in addition to the polypeptide or partial polypeptide thereof, protein or partial protein thereof, nucleic acid, vector, host cell, or host cell population of the present invention.

Examples of the pharmaceutically acceptable carriers include, but are not limited to, excipients such as sucrose, starch, mannitol, sorbitol, lactose, glucose, cellulose, talc, calcium phosphate, calcium carbonate, and the like; binders such as cellulose, methylcellulose, hydroxypropylcellulose, polypropylpyrrolidone, gelatin, gum arabic, polyethylene glycol, sucrose, starch, and the like; disintegrants such as starch, carboxymethylcellulose, hydroxypropyl starch, sodium-glycol-starch, sodium bicarbonate, calcium phosphate, calcium citrate, and the like; lubricants such as magnesium stearate, aerosil, talc, sodium lauryl sulfate, and the like; fragrances such as citric acid, menthol, glycyrrhizin ammonium salt, glycine, orange powder, and the like; preservatives such as sodium benzoate, sodium hydrogen sulfite, methylparaben, propylparaben, and the like; stabilizers such as citric acid, sodium citrate, acetic acid, and the like; suspending agents such as methylcellulose, polyvinylpyrrolidone, aluminum stearate, and the like; dispersing agents such as surfactants and the like; diluents such as water, physiological saline, orange juice, and the like; and base waxes such as cacao butter, polyethylene glycol, white kerosene, and the like.

In order to promote the introduction of the polypeptide or partial polypeptide thereof, protein or partial protein thereof, nucleic acid, and vector of the present invention into cells, the pharmaceutical composition of the present invention may further contain a nucleic acid introduction reagent. As the nucleic acid introduction reagents, cationic lipids such as lipofectin, lipofectamine, DOGS (transfectam), DOPE, DOTAP, DDAB, DHDEAB, HDEAB, polybrene, and poly(ethyleneimine) (PEI), and polysaccharides such as schizophyllan (SPG) can be used. In addition, when a retrovirus is used as an expression vector, retronectin, fibronectin, polybrene, and the like can be used as the introduction reagents.

Examples of dosage unit forms of the pharmaceutical composition of the present invention include liquids, tablets, pills, drinkable liquids, powders, suspensions, emulsions, granules, extracts, fine granules, syrups, infusions, decoctions, eye drops, troches, poultices, liniments, lotions, eye ointments, plasters, capsules, suppositories, enemas, injections (liquids, suspensions, etc.), patches, ointments, jellies, pastes, inhalants, creams, sprays, nasal drops, aerosols, and the like.

The content of the polypeptide or partial polypeptide thereof, protein or partial protein thereof, nucleic acid, or vector of the present invention in the pharmaceutical composition is not particularly limited and may be selected from a wide range. For example, it is about 0.01 to 100% by 25 weight of the entire pharmaceutical composition.

The concentration of the polypeptide or partial polypeptide thereof, protein or partial protein thereof, nucleic acid, or vector of the present invention in the pharmaceutical composition is not particularly limited and may be appropriately selected from a wide range. For example, it is about 0.01 nM to 1 M, preferably 0.1 nM to 10 mM, more preferably 1 nM to 100 nM, of the entire pharmaceutical composition.

When in use, the pharmaceutical composition of the present invention is administered by a method according to various forms. For example, in the case of an injection, it is administered intravenously, intramuscularly, intradermally, subcutaneously, intraarticularly, or intraperitoneally.

The pharmaceutical composition of the present invention is low toxic and can be administered parenterally (e.g., intravascular administration, subcutaneous administration, etc.) to humans or other vertebrates (e.g., mice, rats, rabbits, sheep, pigs, cows, cats, dogs, monkeys, birds, etc.).

The dosage of the pharmaceutical composition of the present invention varies depending on the activity and type of the active ingredient, the mode of administration, the severity of the disease, the animal species to be the administration subject, the drug tolerance, body weight, age, etc. of the administration subject, and cannot be generalized. It is generally about 0.001 mg/kg to about 2.0 g/kg as the active ingredient per day.

Furthermore, the present invention also provides a pharmaceutical composition containing the host cell or host cell population of the present invention. The pharmaceutical composition of the present invention containing the host cell or host cell population of the present invention can be obtained by suspending the host cell or host cell population of the present invention obtained by introducing the nucleic acid or vector of the present invention in physiological saline or an appropriate buffer solution (e.g., phosphate-buffered saline), without limitation. In this case, if the number of the obtained host cells or host cell population is small, they may be cultured and grown until a predetermined cell number is obtained. The obtained host cells or host cell population can be cultured in a normal growth medium, such as, but not limited to, DMEM, EMEM, RPMI-1640, F-12, α-MEM, or MSC growing medium (Bio Whittaker). The culture temperature is usually in the range of about 30 to 40° C., and preferably about 37° C. The CO₂ concentration is usually in the range of about 1 to 10%, and preferably about 5%. The humidity is usually in the range of about 70 to 100%, and preferably about 95 to 100%.

In addition, when using the host cells or host cell population of the present invention in a pharmaceutical composition, dimethyl sulfoxide (DMSO), serum albumin, etc. may be contained in the pharmaceutical composition to protect the host cells or host cell population, and antibiotics, etc. may be contained in the pharmaceutical composition to prevent bacterial contamination and proliferation. Furthermore, other ingredients that are acceptable for formulation (e.g., carriers, excipients, disintegrants, buffers, emulsifiers, suspending agents, soothing agents, stabilizers, preservatives, antiseptics, physiological saline, etc.) may be contained in the pharmaceutical composition. Those skilled in the art can add these factors and drugs to the pharmaceutical composition at appropriate concentrations.

The number of host cells of the present invention contained in the pharmaceutical composition prepared above can be appropriately adjusted in consideration of the sex, age, weight of the subject, condition of the affected area, and condition of the cells to be used, so that the desired effect can be obtained in the treatment of the disease. The subject individual includes, but is not limited to, mammals such as humans. In addition, the pharmaceutical composition of the present invention may be administered multiple times (e.g., 2 to 10 times) at appropriate intervals (e.g., twice a day, once a day, twice a week, once a week, once every two weeks, once a month, once every two months, once every three months, once every six months) until the desired therapeutic effect is obtained. Therefore, depending on the condition of the subject, a therapeutically effective amount may be, for example, 1×10³ cells to 1×10¹⁰ cells per administration per individual, with 1 to 10 administrations. The total amount administered per individual is not limited, but may be 1×10³ cells to 1×10¹¹ cells, preferably 1×10⁴ cells to 1×10¹⁰ cells, further preferably 1×10⁵ cells to 1×10⁹ cells.

The method of administering the pharmaceutical composition containing the host cells or host cell population of the present invention is not particularly limited. Preferred examples include intravascular administration (preferably intravenous administration), intraperitoneal administration, intraintestinal administration, subcutaneous administration, and local administration to the affected area. Among these, intravascular administration and local administration are more preferred examples.

In the pharmaceutical composition of the present invention, the host cells may be either autologous or heterologous (allogeneic or xenogeneic) to the subject to be the administration target, and the individual to be the source of the cells is not particularly limited. For example, when the drug of the present invention is produced for administration to an animal or the like in need of preventing or treating thrombosis, these cells may have tissue compatibility to such an extent that host cells derived from the donor cells can be engrafted in the recipient.

For example, when the pharmaceutical composition of the present invention containing the host cells or host cell population of the present invention is used for preventing or treating thrombosis in a vertebrate, these cells may be the subject's own cells or may be collected from another individual having an HLA type that is identical or substantially identical to the subject's HLA type, from the viewpoint of preventing death due to attack by the immune system. The “substantially the same HLA type” used in the present specification means that the donor's HLA type matches that of the subject to such an extent that host cells derived from the donor's cells can be engrafted when transplanted into a subject using immunosuppressants or the like. For example, HLA types in which the main HLA (e.g., in humans, the three main loci of HLA-A, HLA-B, and HLA-DR, or four loci further including HLA-Cw) are the same can be mentioned.

### 7. Treatment or Prevention

In one embodiment, the pharmaceutical composition of the present invention is for suppressing blood coagulation. Suppression is a concept that includes stopping the progression of blood coagulation to a state where the progression of the disease is stopped, and further, reducing blood coagulation to a state where the disease is cured. In one embodiment, the pharmaceutical composition of the present invention is for treating or preventing thrombosis. Examples of thrombosis that can be treated or prevented with the pharmaceutical composition of the present invention include (congenital or acquired) venous thrombosis, disseminated intravascular coagulation, (neonatal) purpura fulminans, deep vein thrombosis, and (acute or chronic) pulmonary thromboembolism, and thrombosis associated with new coronavirus infection.

In the present specification, the terms “treatment” and “prevention” do not necessarily mean complete treatment or prevention, but are meant to include various degrees of treatment or prevention. Treatment or prevention using the pharmaceutical composition of the present invention may include treatment or prevention of symptoms of a disease. Furthermore, “prevention” may include delaying the onset of a disease or the onset of symptoms thereof, or reducing the possibility of the onset of a disease.

(Gene therapy)

A viral vector containing a nucleic acid encoding the polypeptide or partial polypeptide thereof of the present invention can be prepared by a known method. In brief, a plasmid vector for viral expression is prepared by inserting a nucleic acid encoding the polypeptide or partial polypeptide thereof of the present invention and, where necessary, a nucleic acid having a desired function (e.g., organ-specific promoter, etc.), and this is transfected into an appropriate host cell to transiently produce a viral vector containing the nucleic acid of the present invention, and the viral vector is recovered.

For example, when preparing an AAV vector, first, a vector plasmid is prepared in which the ITRs at both ends of the wild-type AAV genome sequence are left and, in place of the DNA encoding the other Rep protein and capsid protein, a nucleic acid encoding the polypeptide or partial polypeptide thereof of the present invention is inserted. Meanwhile, the DNA encoding the Rep protein and capsid protein required for the formation of virus particles is inserted into another plasmid. Furthermore, a plasmid containing genes (E1A, E1B, E2A, VA, and E4or f6) that are responsible for the adenovirus helper function required for AAV proliferation is produced as an adenovirus helper plasmid. By cotransfecting these three types of plasmids into a host cell, a recombinant AAV (i.e., AAV vector) is produced in the cell. As the host cell, it is preferable to use a cell (e.g., 293 cell, etc.) capable of supplying a part of the gene product (protein) of the aforementioned gene responsible for the helper action. When such a cell is used, it is not necessary to mount a gene encoding a protein that can be supplied by the host cell in the aforementioned adenovirus helper plasmid. Since the produced AAV vector is present in the nucleus, the desired AAV vector is prepared by recovering the host cell by freezing and thawing, and separating and purifying by density gradient ultracentrifugation using cesium chloride, a column method, and the like.

When the pharmaceutical composition of the present invention is used to treat or prevent thrombosis in a subject, the administration route thereof is not particularly limited as long as the protein or partial protein thereof of the present invention, which is the active ingredient, is delivered to the blood. In one embodiment, the composition of the present invention containing a viral vector carrying a nucleic acid encoding the polypeptide or partial polypeptide thereof of the present invention is administered via intramuscular injection.

The composition may also be administered by infusion, transdermal absorption (e.g., via a transdermal patch), inhalation, topical administration to tissue, or may be administered, for example, intravenously, intraperitoneally, orally, intradermally, subcutaneously, or intraarterially.

In one embodiment, the dosage of the viral vector in the composition of the present invention may be about 1×10⁹ to about 6×10¹⁴ vector genomes (vg)/kg, about 1×10¹⁰ to about 4×10¹⁹ vg/kg, about 1×10¹¹ to about 2×10¹⁴ vg/kg, about 1×10¹² to about 1×10¹⁴ vg/kg, or about 5×10¹² to about 1×10¹⁴ vg/kg.

### (Genome Editing Therapy)

The present invention provides a pharmaceutical composition containing a donor vector of the present invention and a vector containing a nucleic acid encoding a nucleic acid metabolic enzyme.

In the present specification, the term “nucleic acid metabolic enzyme” refers to a molecular complex conferred with a particular nucleotide sequence recognition ability and having DNA cleavage activity. The complex may contain a nucleic acid sequence recognition module having DNA cleavage activity, or may contain a nucleic acid sequence recognition module without DNA cleavage activity and a DNA cleavage domain. Here, the “complex” includes not only those composed of multiple molecules, but also those having a nucleic acid sequence recognition module and a DNA cleavage domain in a single molecule, such as a fusion protein. In one embodiment, the nucleic acid metabolic enzyme of the present invention is a nuclease, which includes a protein as a component, or is composed of a protein. In another embodiment, the nuclease of the present invention includes a component (e.g., nucleic acid) other than a protein.

In the present invention, the “nucleic acid sequence recognition module” refers to a molecule or molecular complex having the ability to specifically recognize and bind to a particular nucleotide sequence on a DNA strand (i.e., target nucleotide sequence). The binding of the nucleic acid sequence recognition module to a target nucleotide sequence enables the module or the DNA cleavage domain linked to the module to act specifically on a targeted site in DNA. In one embodiment, the “nucleic acid sequence recognition module” itself has DNA cleavage activity. In another embodiment, the “nucleic acid sequence recognition module” itself does not have DNA cleavage activity.

In the present invention, the “DNA cleavage domain” refers to a polypeptide that catalyzes a reaction that cleaves one or both strands of the double helix constituting DNA. Examples of such polypeptides include polypeptide of the restriction enzyme FokI and the like.

The target nucleotide sequence in DNA recognized by the nucleic acid sequence recognition module is not particularly limited as long as the module can specifically bind thereto, and may be any sequence in DNA. The length of the target nucleotide sequence may be any as long as it is sufficient for the nucleic acid sequence recognition module to specifically bind thereto, and is, for example, 12 nucleotides or more, preferably 15 nucleotides or more, more preferably 17 nucleotides or more. There is no particular upper limit to the length, but it is preferably 25 nucleotides or less, more preferably 22 nucleotides or less.

In one embodiment of the present invention, the nucleic acid sequence recognition module is a CRISPR-Cas system. In this embodiment, since the nucleic acid sequence recognition module itself has DNA cleavage activity, it is not necessarily required to form a complex of the nucleic acid sequence recognition module and a DNA cleavage domain.

The above-mentioned CRISPR-Cas system recognizes the sequence of the target donor DNA (double strand DNA or single strand DNA) by a guide RNA having a target nucleotide sequence (here, RNA sequence). Thus, any sequence can be targeted by simply synthesizing an oligo DNA that can specifically hybridize with the complementary sequence of the target nucleotide sequence.

The CRISPR/Cas system has the activity of also recognizing single strand DNA as a substrate and cleaving same (Ma, E., Mol. Cell, (2015) 60(3), 398-407). In one embodiment, the donor DNA can be a double strand DNA that contains a single strand DNA. In homologous recombination, the cut end of the donor DNA is trimmed to expose the single strand DNA, and the single strand binds to a homologous sequence site on the chromosome. Therefore, the donor DNA may be more efficiently incorporated into the chromosome by homologous recombination.

The nucleic acid sequence recognition module using CRISPR-Cas is provided as a complex of a target nucleotide sequence (here, RNA sequence), an RNA molecule (guide RNA) consisting of tracrRNA required for recruiting Cas protein, and the Cas protein. In another embodiment, the nucleic acid sequence recognition module using CRISPR-Cas is provided as a complex of crRNA containing RNA having the same sequence as the target nucleotide sequence, tracrRNA, and Cas.

The Cas protein used in the present invention is not particularly limited as long as it belongs to the CRISPR system, and examples thereof include Cas9 and Cpf1, with preference given to Cas9. Examples of Cas9 include, but are not limited to, Cas9 (SpCas9) derived from Streptococcus pyogenes, Cas9 (StCas9) derived from Streptococcus thermophilus, and Cas9 (SaCas9) derived from Staphylococcus aureus. SpCas9 is preferred.

When CRISPR-Cas is used as a nucleic acid sequence recognition module, it is desirable to introduce the nucleic acid sequence recognition module into a cell in the form of a nucleic acid (expression vector) that encodes the module. That is, an expression vector encoding a guide RNA and a Cas protein is introduced into a cell, and the guide RNA and the Cas protein are expressed, thereby forming a complex of the guide RNA and the Cas protein in the cell. The guide RNA and the Cas protein may be encoded on the same expression vector, or may be encoded on different expression vectors.

The DNA encoding Cas can be cloned by a method well known in the art from a cell that produces Cas.

The obtained DNA encoding Cas can be inserted downstream of the promoter of an expression vector appropriate for the host.

On the other hand, the DNA encoding the guide RNA can be chemically synthesized using a DNA/RNA synthesizer by designing an oligo DNA sequence that links a target nucleotide sequence (here, RNA sequence) and a known tracrRNA sequence. DNA encoding the guide RNA can also be inserted into an expression vector appropriate for the host. The guide RNA and Cas may be encoded on the same expression vector, or may be encoded on different expression vectors. Preferably, the DNA encoding Cas and the DNA encoding the guide RNA and tracrRNA are inserted downstream of separate promoters in the same expression vector.

As the target nucleotide sequence in the present invention, a sequence adjacent (on the 5′ or 3′ side) to a PAM sequence in a sequence contained in the host cell genome is selected. In one embodiment, the Cas protein is SpCas9, and a sequence adjacent immediately before the 5′ side of the PAM sequence (5′-NGG) in a sequence contained in the host cell genome is selected. In another embodiment, the Cas protein is SaCas9, and a sequence adjacent immediately before the 5′ side of the PAM sequence (NNGRR(T)) in a sequence contained in the host cell genome is selected. In yet another embodiment, the PAM sequence can be 5′-NG or 5′-NNG. The target nucleotide sequence of other Cas (Cas12 genus) is a sequence on the 3′ side of the PAM sequence.

The sequence in the donor DNA (e.g., donor vector) used in the present invention that is homologous to the sequence before and after the site in the genome sequence in which the nucleic acid of the present invention is incorporated by homologous recombination includes the above-mentioned target nucleotide sequence.

The RNA encoding Cas can be prepared, for example, by using the above-mentioned DNA encoding Cas as a template and transcribing same into mRNA in a known in vitro transcription system.

The guide RNA can be chemically synthesized using a DNA/RNA synthesizer by designing an oligoRNA sequence that links the target nucleotide sequence (here, RNA sequence) with a known tracrRNA sequence.

In the present specification, the nucleotide sequence is described as the sequence of DNA unless otherwise specified. When the polynucleotide is RNA, thymine (T) is appropriately replaced with uracil (U).

In another embodiment of the present invention, the nucleic acid sequence recognition module that can be used may be a zinc finger motif (Japanese Patent No. 4968498), a TAL effector (Japanese Patent Publication No. 2013-513389), a PPR motif (Japanese Patent Publication No. 2013-128413), or a fragment containing a DNA-binding domain of a protein that can specifically bind to DNA, such as a restriction enzyme, a transcription factor, or an RNA polymerase, and not having the ability to cleave DNA double strands.

Tha The above-mentioned nucleic acid sequence recognition module may be provided as a fusion protein with the above-mentioned DNA cleavage domain, or a protein binding domain such as an SH3 domain, a PDZ domain, a GK domain, or a GB domain and binding partners thereof may be fused to the nucleic acid sequence recognition module and the DNA cleavage domain, respectively, and provided as a protein complex via the interaction between the protein binding domain and the binding partner thereof. Alternatively, an intein may be fused to each of the nucleic acid sequence recognition module and the DNA cleavage domain, and the two can be linked by ligation after the synthesis of each protein.

The above-mentioned nuclease can be contacted with the genomic double strand DNA and the above-mentioned donor DNA (e.g., donor vector) by introducing a nucleic acid encoding the nuclease into a cell together with the above-mentioned donor DNA (e.g., donor vector).

Therefore, it is preferable to prepare the nucleic acid sequence recognition module, or the nucleic acid sequence recognition module and the DNA cleavage domain, as a nucleic acid encoding a fusion protein thereof, or as a nucleic acid encoding each component in a form capable of forming a complex in a host cell after translation into a protein. Here, the nucleic acid may be either DNA or RNA. In the case of DNA, it is preferably double strand DNA, and is provided in the form of an expression vector capable of expressing each component under the control of a functional promoter in a host cell. In the case of RNA, it is preferably single strand RNA.

DNAs encoding nucleic acid sequence recognition modules such as zinc finger motifs, TAL effectors, and PPR motifs can be obtained by any of the methods described in the above-mentioned documents with respect to each module. DNAs encoding sequence recognition modules such as restriction enzymes, transcription factors, and RNA polymerases can be cloned, for example, by synthesizing an oligo-DNA primer based on the cDNA sequence information thereof so as to cover a region encoding a desired portion of the protein (portion including DNA-binding domain), and amplifying the primer by RT-PCR using total RNA or an mRNA fraction prepared from cells that produce the protein as a template.

DNAs encoding DNA cleavage domains can also be cloned by synthesizing an oligo-DNA primer based on the cDNA sequence information of the domain to be used, and amplifying the primer by RT-PCR using total RNA or an mRNA fraction prepared from cells that produce the domain as a template. For example, the DNA encoding FokI can be cloned from mRNA derived from Flavobacterium okeanokoites (IFO 12536) by RT-PCR, using appropriate primers designed for the upstream and downstream of the CDS based on the cDNA sequence.

The cloned DNA can be ligated as it is or after digestion with a restriction enzyme or addition of an appropriate linker and/or nuclear/organelle localization signal when desired, to the DNA encoding the nucleic acid sequence recognition module to prepare a DNA encoding a fusion protein. Alternatively, the DNA encoding the nucleic acid sequence recognition module and the DNA encoding the DNA cleavage domain may be each fused to DNA encoding a binding domain or a binding partner thereof, or both DNAs may be fused to DNA encoding a separation intein, so that the nucleic acid sequence recognition module and the DNA cleavage domain can form a complex after translation in the host cell. In these cases, a linker and/or a nuclear localization signal can be linked to an appropriate position in one or both DNAs when desired.

An expression vector containing DNA encoding a nucleic acid sequence recognition module and/or a DNA cleavage domain can be produced, for example, by linking the DNA to the downstream of a promoter in an appropriate expression vector. When the CRISPR-Cas system is used as the nucleic acid sequence recognition module, an expression vector encoding a guide RNA and a Cas protein is introduced into a host cell, and the guide RNA and the Cas protein are expressed to form a complex of the guide RNA and the Cas protein in the host cell. The guide RNA and the Cas protein may be encoded on the same expression vector, or may be respectively encoded on different expression vectors.

In addition to the above, the expression vector may contain enhancer, splicing signal, terminator, polyA addition signal, selection marker such as drug resistance gene and auxotrophy complementation gene, replication origin, and the like, when desired.

The RNA encoding the nucleic acid sequence recognition module and/or the DNA cleavage domain can be prepared, for example, by using a vector encoding the DNA encoding the above-mentioned nucleic acid sequence recognition module and/or the DNA cleavage domain as a template and transcribing same into mRNA in a known in vitro transcription system.

In one embodiment, a pharmaceutical composition containing the donor vector of the present invention and a vector containing a nucleic acid encoding a nucleic acid metabolic enzyme is administered to a subject, and the genomic double strand DNA containing a selected target nucleotide sequence in the cells of the subject and the above-mentioned donor vector are contacted with a nuclease that cleaves DNA within the target nucleotide sequence.

By introducing the above-mentioned donor DNA (e.g., donor vector) and an expression vector encoding the components of the above-mentioned nuclease (nucleic acid sequence recognition module and/or DNA cleavage domain) into a cell, the above-mentioned nuclease is formed in the host cell, and the nuclease can be contacted with the genomic double strand DNA and the above-mentioned donor vector.

In another embodiment, a pharmaceutical composition including RNA encoding a nucleic acid sequence recognition module and/or a DNA cleavage domain and a guide RNA is administered to a subject, and the genomic double strand DNA including a selected target nucleotide sequence in the cells of the subject and the above-mentioned donor vector are contacted with a nuclease that cleaves DNA within the target nucleotide sequence. The pharmaceutical composition can be embedded in a lipid nanoparticle (LNP) and introduced into the cell.

By introducing the above-mentioned donor DNA (e.g., donor vector) and RNA encoding the components of the above-mentioned nuclease (nucleic acid sequence recognition module and/or DNA cleavage domain) into a cell, the above-mentioned nuclease is formed in the host cell, and the nuclease can be contacted with the genomic double strand DNA and the above-mentioned donor vector.

Introduction of donor DNA (e.g., donor vector) into cells can be carried out according to known methods (e.g., lysozyme method, competent method, PEG method, CaCl₂ coprecipitation method, electroporation method, microinjection method, particle gun method, lipofection method, Agrobacterium method, etc.) depending on the type of cell. When a donor vector and an expression vector containing DNA encoding a nucleic acid sequence recognition module and/or DNA cleavage domain (expression vector encoding guide RNA and Cas protein when using the CRISPR-Cas system as the nucleic acid sequence recognition module) are viral vectors, they can be directly administered (e.g., intravenous administration) to a subject and introduced into the cells of the subject.

The number of molecules of the above-mentioned donor DNA (e.g., donor vector) used in the introduction operation, when calculated as the number of copies of a homologous nucleotide sequence per host cell, is, for example, 1×10² to 1×10⁸ molecules, preferably 4×10³ to 4×10⁴ molecules.

In one embodiment, when the donor vector is a viral vector, the dosage of the viral vector in the composition of the present invention may be about 1×10⁹ to about 6×10¹⁴ vector genomes (vg)/kg, about 1×10¹⁰ to about 4×10¹⁴ vg/kg, about 1×10¹¹ to about 2×10¹⁴ vg/kg, about 1×10¹² to about 1×10¹⁴ vg/kg, or about 5×10¹² to about 1×10¹⁴ vg/kg.

The number of molecules of the expression vector encoding the components of the above-mentioned nuclease (nucleic acid sequence recognition module and/or DNA cleavage domain) used in the introduction operation is, for example, 1×10² to 1×10⁹ molecules, preferably 4×10⁴ to 4×10⁵ molecules per host cell.

When the CRISPR-Cas system is used as the nucleic acid sequence recognition module and the expression vector for the Cas protein and the expression vector for the guide RNA are different, the ratio of the number of molecules of those expression vectors to be introduced is, for example, 1:0.4 to 1:1.6, preferably 1:0.5 to 1:1.5.

In one embodiment, when the expression vector encoding the components of the above-mentioned nuclease (nucleic acid sequence recognition module and/or DNA cleavage domain) is a viral vector, the dosage of the viral vector in the composition of the present invention may be about 1×10⁹ to about 6×10¹⁴ vector genomes (vg)/kg, about 1×10¹⁰ to about 4×10¹⁴ vg/kg, about 1×10¹¹ to about 2×10¹⁴ vg/kg, about 1×10¹² to about 1×10¹⁴ vg/kg, or about 5×10¹² to about 1×10¹⁴ vg/kg.

When a nucleic acid sequence recognition module or a complex of a nucleic acid sequence recognition module and a DNA cleavage domain (nuclease) is expressed from a nucleic acid or expression vector introduced into a cell, the nucleic acid sequence recognition module specifically recognizes and binds to a target nucleotide sequence in a donor DNA (e.g., donor vector) and/or a genomic double strand DNA, and the DNA is cleaved at a targeted site (which can be appropriately adjusted within a range of several hundred bases including all or part of the target nucleotide sequence or vicinity thereof) by the action of the nucleic acid sequence recognition module itself or the DNA cleavage domain linked to the nucleic acid sequence recognition module. In one embodiment, the nuclease preferentially cleaves a target nucleotide sequence in a nucleotide sequence that is homologous to a genomic sequence contained in the donor DNA (e.g., donor vector). Thereafter, a repair mechanism known as homologous recombination (directed) repair (HDR), which exists in almost all cell types and organisms, causes homologous recombination between the sequence on the genomic double strand DNA and the homologous nucleotide sequence contained in the donor DNA (e.g., donor vector), and the DNA sequence encoding the polypeptide or partial polypeptide thereof of the present invention contained in the donor DNA (e.g., donor vector) is inserted into the targeted site of the sequence on the genomic double strand DNA. The polypeptide or partial polypeptide thereof of the present invention is then expressed, and the protein or partial protein of the present invention is formed. The protein or partial protein of the present invention exhibits blood anticoagulant action and can be used to suppress blood coagulation and treat or prevent diseases (thrombosis, etc.).

In one embodiment, when a linear double strand DNA is used as the donor DNA, the linear double strand DNA can be obtained by cleaving the above-mentioned circular double strand DNA at the target nucleotide sequence to form a linear DNA. After the linear donor DNA is introduced into a host cell, a repair mechanism known as homologous recombination (directed) repair (HDR), which exists in almost all cell types and organisms, causes homologous recombination between the sequence on the genomic double strand DNA and the homologous nucleotide sequence contained in the donor DNA of the linear double strand DNA, and the DNA sequence encoding the polypeptide or partial polypeptide thereof of the present invention contained in the donor DNA (e.g., circular double strand DNA) is inserted into the targeted site on the genomic double strand DNA.

### 8. Preparation

The polypeptide of the present invention may be further imparted with a signal peptide. Wild-type protein C is translated in cells as wild-type human protein C prepropolypeptide in which the signal peptide is linked to the N-terminus of the amino acid sequence represented by SEQ ID NO: 2, and the above-mentioned signal peptide is cleaved when it is secreted outside the cell, resulting in conversion into a pro-type protein. When the polypeptide or partial polypeptide thereof of the present invention is expressed in a cell in order to produce a recombinant protein, since the signal peptide is added, it is secreted outside the cell to facilitate recovery thereof.

The polypeptide or partial polypeptide thereof of the present invention can be produced according to a known peptide synthesis method.

The peptide synthesis method may be, for example, either a solid-phase synthesis method or a liquid-phase synthesis method. The target polypeptide or partial polypeptide thereof can be produced by condensing a partial peptide or amino acid capable of constituting the polypeptide or partial polypeptide thereof of the present invention with the remaining portion, and by removing a protecting group when the product has the protecting group.

The condensation and removal of the protecting group are carried out according to a method known per se, for example, 15 the methods described in (1) and (2) below.
(1) M. Bodanszky and M. A. Ondetti, Peptide Synthesis, Interscience Publishers, New York (1966)
(2) Schroeder and Luebke, The Peptide, Academic Press, New York (1965)

The thus-obtained polypeptide or partial polypeptide thereof of the present invention can be purified and isolated by a known purification method. Examples of the purification method include solvent extraction, distillation, column 25 chromatography, liquid chromatography, recrystallization, and combinations of these.

When the polypeptide or partial polypeptide thereof of the present invention obtained by the above-mentioned method is in a free form, the free form can be converted into an appropriate salt by a known method or a method analogous thereto. Conversely, when the polypeptide or partial polypeptide thereof of the present invention is obtained as a salt, the salt can be converted into a free form or another salt by a known method or a method analogous thereto.

The polypeptide of the present invention can also be produced using a cell-free protein synthesis system. In producing the polypeptide or partial polypeptide thereof of the present invention, RNA transcribed from DNA containing the nucleic acid of the present invention can be used as a translation template, or DNA containing the nucleic acid of the present invention can be used as a transcription template for producing a translation template in vitro. In addition to the polynucleotide of the present invention, the translation template can contain an RNA polymerase recognition sequence (e.g., SP6, T3, or T7 promoter) and a sequence that enhances the translation activity in the synthesis system (e.g., Q sequence or E01 sequence). As a cell-free protein synthesis system, a method well known to those skilled in the art, such as the method described in International Publication No. 05/030954 using wheat germ extract, can be appropriately used.

The protein or partial protein thereof of the present invention can also be produced by culturing a host cell containing an expression vector containing a nucleic acid encoding the polypeptide or partial polypeptide thereof of the present invention, and isolating and purifying the protein or partial protein thereof of the present invention from the resulting culture.

The polypeptide or partial polypeptide thereof, or the protein or partial protein thereof of the present invention, can be separated and purified according to a method known per se, from the culture obtained by the aforementioned cell-free protein synthesis system or culturing the gene-introduced host cells.

For example, when extracting the polypeptide or partial polypeptide thereof, or the protein or partial protein thereof of the present invention from the cell-free protein synthesis system or the host cells, a method is appropriately used in which the host cells collected from the culture by a known method are suspended in an appropriate buffer solution, and the host cells are disrupted as necessary by ultrasonic waves, lysozyme and/or freezing and thawing, and then a crude extract of the soluble protein is obtained by centrifugation or filtration. The buffer solution may contain a protein denaturant such as urea or guanidine hydrochloride, or a surfactant such as Triton X-100™ and the like. Furthermore, when the polypeptide or partial polypeptide thereof, or the protein or partial protein thereof of the present invention is secreted outside the cells, a method is used in which the culture supernatant is separated from the culture by centrifugation, filtration, or the like.

The mutant AIM of the present invention contained in the soluble fraction and culture supernatant thus obtained can be isolated and purified according to a method known per se. Examples of such methods include methods that utilize solubility, such as salting out and solvent precipitation; methods that mainly utilize differences in molecular weight, such as dialysis, ultrafiltration, gel filtration, and SDS-polyacrylamide gel electrophoresis; methods that utilize differences in charge, such as ion exchange chromatography; methods that utilize specific affinity, such as affinity chromatography; methods that utilize differences in hydrophobicity, such as reversed-phase high performance liquid chromatography; methods that utilize differences in isoelectric point, such as isoelectric focusing; and methods that use antibodies. These methods can also be combined as appropriate.

In one embodiment, in order to facilitate the purification of the polypeptide or partial polypeptide thereof, or the protein or partial protein thereof of the present invention, a tag sequence for purification can be inserted into the propeptide or between the signal peptide and the propeptide. Examples of such tag sequences include, but are not limited to, a histidine tag, a maltose binding protein (MBP) tag, and a glutathione S-transferase (GST) tag. The polypeptide or partial polypeptide thereof, or the protein or partial protein thereof of the present invention, into which a tag sequence for purification has been inserted, can be easily separated and purified by passing the culture supernatant of transfectant mammalian cells through a column packed with a ligand that interacts with the tag sequence depending on the type of tag sequence (e.g., in the case of a histidine tag, a column to which a divalent metal ion such as nickel or cobalt is immobilized). The polypeptide or partial polypeptide thereof, or the protein or partial protein thereof of the present invention adsorbed to the column can be purified by passing an eluent having an appropriate salt concentration through the column.

The tag sequence for purification can be chemically synthesized based on known amino acid sequence information, and linked to DNA encoding the signal codon and propeptide by restriction enzyme treatment or using an appropriate linker. Alternatively, DNA encoding a chimeric protein consisting of tag sequence-propeptide or signal peptide-tag sequence-propeptide can be constructed in the same manner as above by combining chemical synthesis with PCR or Gibson Assembly.

The presence of the thus-obtained polypeptide or partial polypeptide thereof, or the protein or partial protein thereof of the present invention can be confirmed by enzyme immunoassay or Western blotting using an antibody against the polypeptide or partial polypeptide thereof, or the protein or partial protein thereof of the present invention. The polypeptide or partial polypeptide of the present invention can be made into the protein or partial protein thereof of the present invention by cleavage of the self-cleaving site and, if necessary, further processing. The polypeptide or partial polypeptide of the present invention can be made into the protein or partial protein thereof of the present invention in vitro by an appropriate protease treatment.

The present invention provides activated protein C isolated and purified from protein C-expressing cells produced by the above-mentioned method for producing protein C-expressing cells of the present invention. The present invention also provides a recombinant preparation of protein C in which the activated protein C is formulated.

### EXAMPLES

The present invention is described in more detail below with reference to examples; however, the present invention is not limited to these examples.

### Example 1

Wild-type PROC cDNA was obtained by reverse transcription PCR (RT-PCR) of human liver RNA. Codon-optimized PROC cDNA was synthesized using the GenScript or GeneArt™ artificial gene synthesis algorithm. Gene was inserted using InFusion Cloning (Takara) after artificially synthesizing the insertion sequence.

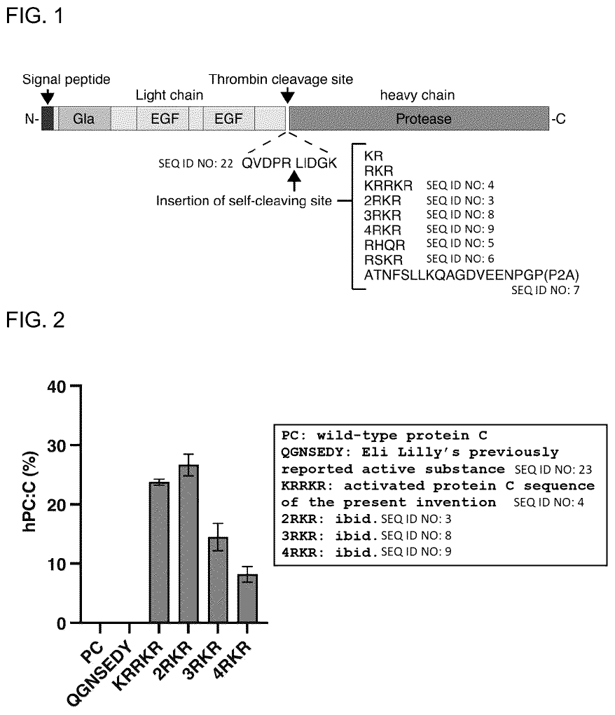

Codon-optimized sequences were used in cell experiments, and wild-type sequences were used in mouse experiments. A schematic diagram of the polypeptide encoded by the cDNA is shown in FIG. 1. Gene sequences used (when the self-cleaving site is RKRRKR; sequences other than the self-cleaving site are common to each construct)

Wild-type PC-2RKR (RKRRKR site is underlined)
(SEQ ID NO: 17)
atgtggcagctcacaagcctcctgctgttcgtggccacctggggaatttccggcacaccagct
cctcttgactcagtgttctccagcagcgagcgtgcccaccaggtgctgcggatccgcaaacgt
gccaactccttcctggaggagctccgtcacagcagcctggagcgggagtgcatagaggagatc
tgtgacttcgaggaggccaaggaaattttccaaaatgtggatgacacactggccttctggtcc
aagcacgtcgacggtgaccagtgcttggtcttgcccttggagcacccgtgcgccagcctgtgc
tgcgggcacggcacgtgcatcgacggcatcggcagcttcagctgcgactgccgcagcggctgg
gagggccgcttctgccagcgcgaggtgagcttcctcaattgctcgctggacaacggcggctgc
acgcattactgcctagaggaggtgggctggcggcgctgtagctgtgcgcctggctacaagctg
ggggacgacctcctgcagtgtcaccccgcagtgaagttcccttgtgggaggccctggaagcgg
atggagaagaagcgcagtcacctgaaacgagacacagaagaccaagaagaccaagtagatccg
cggaggaagcggagaaagcggctcattgatgggaagatgaccaggcggggagacagcccctgg
caggtggtcctgctggactcaaagaagaagctggcctgcggggcagtgctcatccacccctcc
tgggtgctgacagcggcccactgcatggatgagtccaagaagctccttgtcaggcttggagag
tatgacctgcggcgctgggagaagtgggagctggacctggacatcaaggaggtcttcgtccac
cccaactacagcaagagcaccaccgacaatgacatcgcactgctgcacctggcccagcccgcc
accctctcgcagaccatagtgcccatctgcctcccggacagcggccttgcagagcgcgagctc
aatcaggccggccaggagaccctcgtgacgggctggggctaccacagcagccgagagaaggag
gccaagagaaaccgcaccttcgtcctcaacttcatcaagattcccgtggtcccgcacaatgag
tgcagcgaggtcatgagcaacatggtgtctgagaacatgctgtgtgcgggcatcctcggggac
cggcaggatgcctgcgagggcgacagtggggggcccatggtcgcctccttccacggcacctgg
ttcctggtgggcctggtgagctggggtgagggctgtgggctccttcacaactacggcgtttac
accaaagtcagccgctacctcgactggatccatgggcacatcagagacaaggaagccccccag
aagagctgggcaccttag
Codon-optimized PC-2RKR (RKRRKR site is underlined)
(SEQ ID NO: 18)
atgtggcagctgacttcactgctgctgtttgtcgctacttggggaattagtggaactcctgct
cctctggactctgtcttctcctctagcgagagagcccaccaggtgctgaggatccgcaagcgg
gccaactccttcctggaggagctgagacacagctccctggagagggagtgcatcgaggagatc
tgtgacttcgaggaggccaaggagatctttcagaatgtggacgataccctggccttttggtcc
aagcacgtggacggcgatcagtgcctggtgctgccactggagcacccctgtgcctctctgtgc
tgtggccacggcacatgcatcgacggcatcggctccttctcttgcgattgtaggtccggatgg
gagggccgcttctgccagagagaggtgtcttttctgaactgtagcctggataatggcggatgc
acccactactgtctggaggaagtgggatggcggagatgctcctgtgcaccaggctataagctg
ggcgacgatctgctgcagtgccacccagccgtgaagtttccttgtggcagaccatggaagagg
atggagaagaagcgcagccacctgaagcgggacaccgaggatcaggaggaccaggtggatcct
cgcaggaagcggagaaagcggctgatcgacggcaagatgacaagacgcggcgatagcccatgg
caggtggtgctgctggacagcaagaagaagctggcatgtggagccgtgctgatccacccatcc
tgggtgctgacagccgcccactgtatggacgagtctaagaagctgctggtgcggctgggcgag
tacgatctgcggagatgggagaagtgggagctggacctggatatcaaggaggtgttcgtgcac
cccaactatagcaagtccaccacagacaatgatatcgccctgctgcacctggcacagcctgcc
accctgtctcagacaatcgtgcctatctgtctgcctgactctggcctggcagaaagagagctg
aaccaggcaggacaggagacactggtgacaggctggggctaccactctagccgggagaaggag
gccaagagaaaccggaccttcgtgctgaacttcatcaagatccccgtggtgcctcacaatgag
tgctctgaagtgatgagcaacatggtgtccgagaatatgctgtgcgccggcatcctgggcgac
agacaggatgcatgtgaaggcgattccggcggccctatggtggcatctttccacggcacctgg
tttctggtgggcctggtgtcttggggcgagggatgtggcctgctgcacaactacggcgtgtat
acaaaggtgagcaggtatctggactggattcacgggcacattagagacaaagaagcacctcag
aagagttgggcaccataa
Codon-optimized PC-2RKR (RKRRKR site is underlined)
(SEQ ID NO: 18)

### Example 2

Various PROC cDNAs were inserted into pCDNA3 plasmid, and transfection into HEK293 cells derived from human fetal kidney cells was performed using Lipofectamine (registered trademark) 3000 (Thermo Fisher Scientific, Waltham, MA, USA). Gene-expressing cells were selected using G418. Vitamin K (K2N 5 μg/ml) was added to the supernatant, and the cell supernatant was collected after 24 hr. Human PC activity was measured using Verichrome Protein C (Sysmex, Kobe, Japan) with a fully automated blood coagulation measuring device CS1600 (Sysmex). PC activity was evaluated by cleavage of substrate under the conditions without the snake venom (activator) included in the kit. The results are shown in FIG. 2. PC activity increased when the self-cleaving site was KRRKR, 2RKR (RKRRKR), 3RKR (RKRRKRRKR), and 4RKR (RKRRKRRKRRKR).

### Example 3

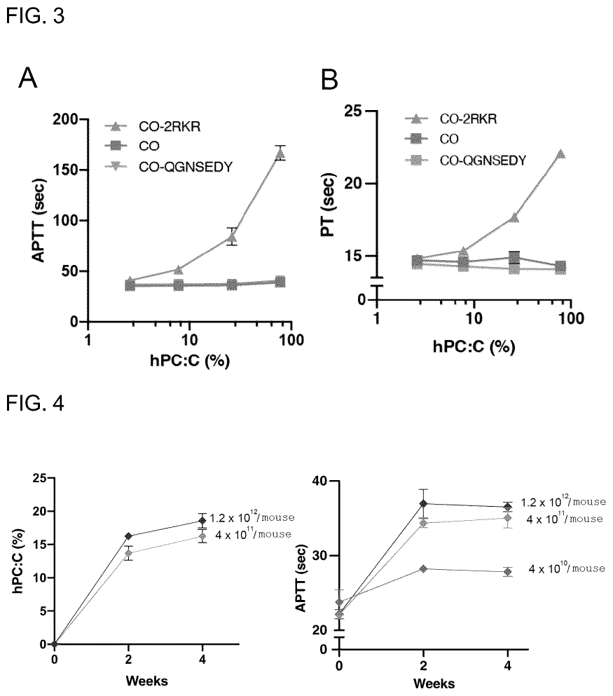

Various PROC cDNAs were inserted into pCDNA3 plasmid, and gene transfer into HEK293 cells was performed using Lipofectamine (registered trademark) 3000. Gene-expressing cells were selected using G418. Vitamin K (K2N 5 μg/ml) was added to the supernatant, and the cell supernatant was collected after 24 hr, and PC activity was measured under conditions containing an activator. The cell supernatant was diluted so that the PC activity was about 0%, 2.6, 8%, 26%, and 80%, and mixed with an equal amount of human standard plasma (Sysmex). The coagulation time, Activated Partial Thromboplastin Time (Thrombocheck APTT, Sysmex) (A) and prothrombin time (Thrombocheck PT, Sysmex) (B) were measured using CS510 (Sysmex). The results are shown in FIG. 3. When the self-cleaving site was 2RKR (RKRRKR), a concentration-dependent extension of the coagulation time was observed.

### Example 4

PROC cDNA (2RKR) was inserted between the liver-specific HCRhAAT promoter and the SV40 polyA sequence. The HCRhAAT promoter is composed—of the Apo E/C1 hepatic control region and the human α1 antitrypsin promoter. This sequence was inserted into a plasmid containing the AAV ITR. AAV8 type vectors were prepared by plasmid transfection method using a helper-free system in the same manner as in previous studies (Ohmori T, Nagao Y, Mizukami H, Sakata A. Muramatsu SI, Ozawa K, et al. CRISPR/Cas9-mediated genome editing via postnatal administration of AAV vector cures haemophilia B mice. Sci Rep. 2017; 7(1):4159). The inverted terminal repeat (ITR) of the AAV vector used was derived from AAV2 type. The titer of the purified AAV vector was measured by quantitative PCR (qPCR). AAV8 vectors were administered intravenously to wild-type C57BL/6 mice (7-8 weeks old, male) via the jugular vein under isoflurane anesthesia (4×10¹⁰, 4×10¹¹, 1.2×10¹² vg/mouse). Blood was collected 2 and 4 weeks after vector administration, and plasma PC activity and APTT were measured. The results are shown in FIG. 4. An increase in human PC activity and prolongation of APTT were observed in mouse blood when vector was administered.

### Example 5

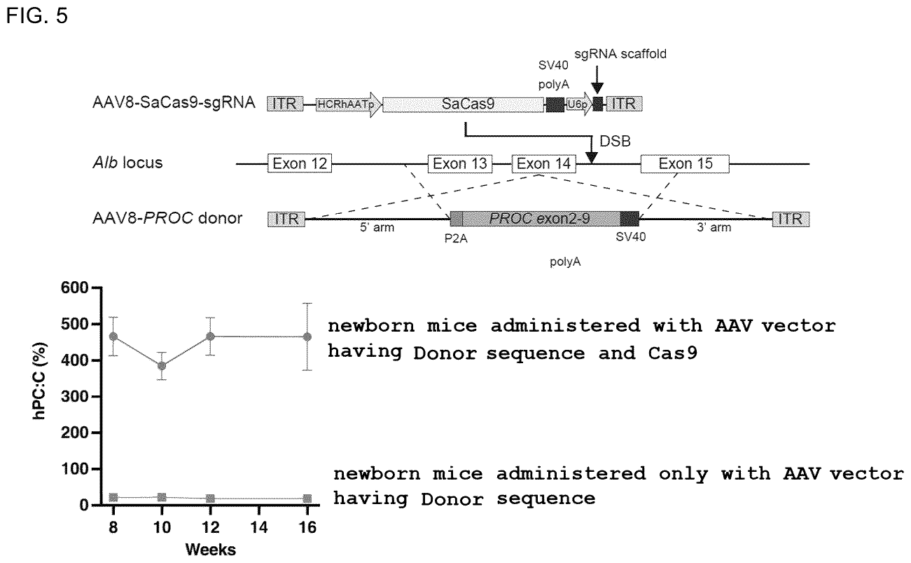

According to previous reports (De Caneva A, Porro F, Bortolussi G, Sola R, Lisjak M, Barzel A, et al. Coupling AAV-mediated promoterless gene targeting to SaCas9 nuclease to efficiently correct liver metabolic diseases. JCI Insight. 2019; 4(15):e128863. https://doi.org/10.1172/jci.insight.128863.), a guide RNA was designed in the mouse Alb locus, and an AAV8 type vector (Cas9) expressing SaCas9 and an AAV8 type vector (Donor) having a wild-type PC sequence bound to a P2A sequence having a homologous recombination sequence (about 1 kb) at both ends were administered to wild-type C57BL/6 newborn mice. The results are shown in FIG. 5. Human PC activity continuously increases in newborn mice administered with an AAV vector having a Donor sequence and Cas9. PC activity does not increase with the Donor sequence alone.

### Example 6

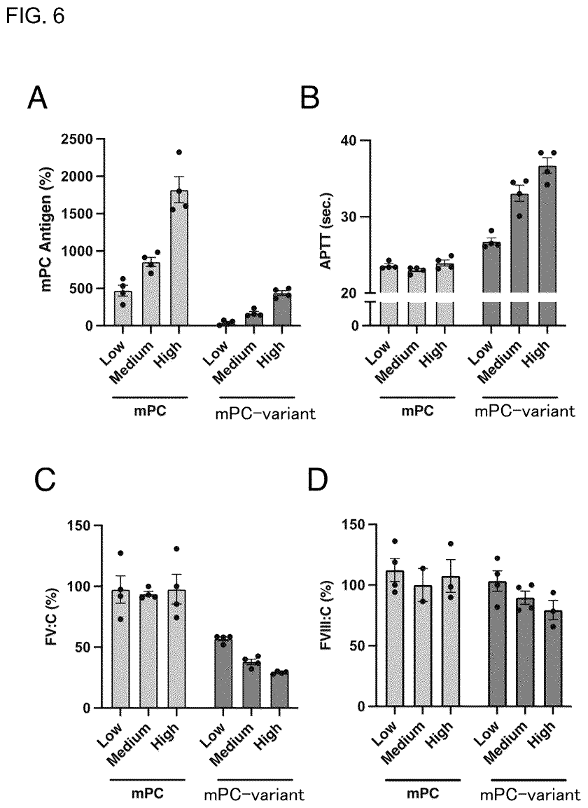

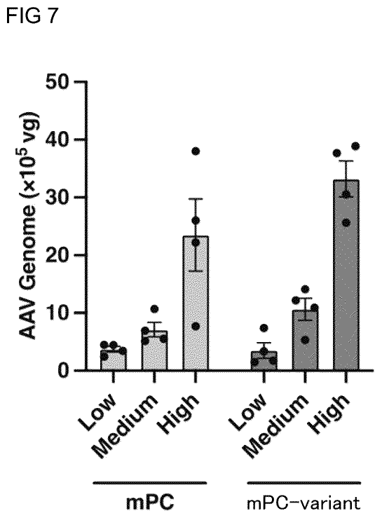

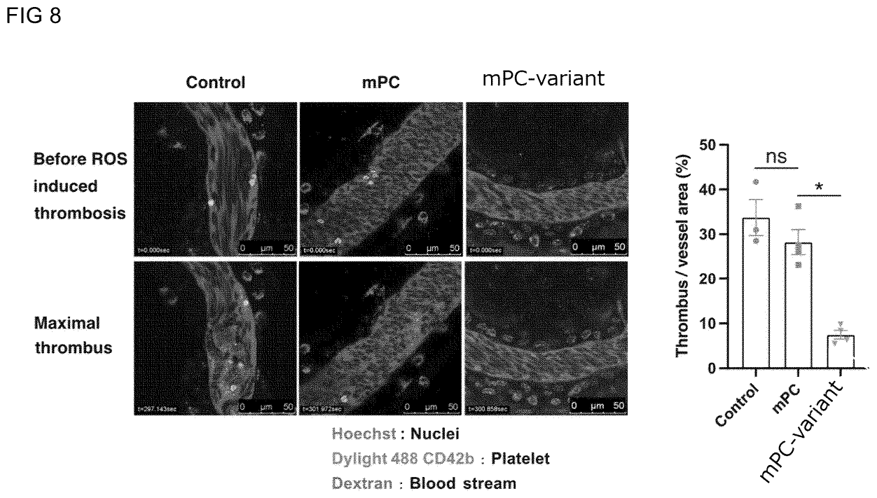

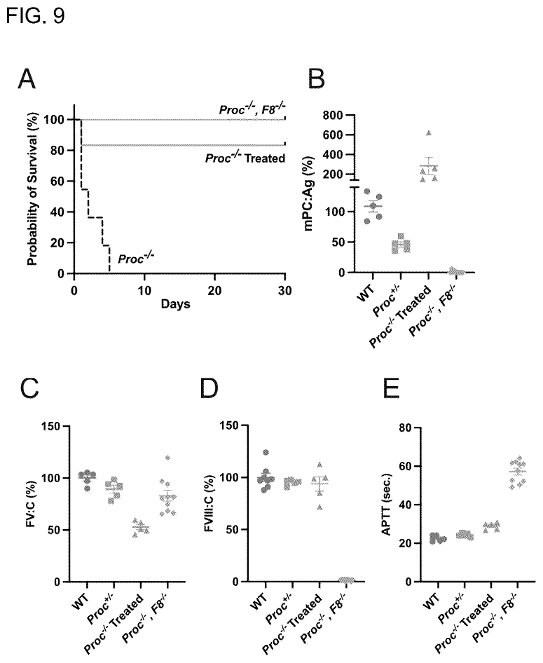

To wild-type C57BL/6 mice (7 weeks old, male) were intravenously administered a single dose of AAV8 type vector expressing wild-type mouse protein C sequence (mPC) or modified mouse protein C sequence (mPC variant) at three doses of Low (4×10¹⁰ vg/mouse), Medium (1.2×10¹¹ vg/mouse), and High (4×10¹¹ vg/mouse). Blood was collected from the mice 4 to 8 weeks after administration, and plasma was obtained. The increase in protein C antigen level in plasma (FIG. 6A), coagulation time [activated partial thromboplastin time (APTT)](FIG. 6B), factor V activity (FIG. 6C), and factor VIII activity (FIG. 6D) were measured according to conventional methods. Only the mPC variant showed a vector dose-dependent suppression of blood coagulation. The amount of AAV genome in the liver (FIG. 7) did not change in any group. When pathological thrombus formation dependent on reactive oxygen was confirmed in mouse testicular veins, the formation of pathological thrombus was suppressed in the mPC variant (examination only in Low group, FIG. 8).

The evaluation method for each index is as follows.
Mouse protein C antigen amount: A 96-well plate on which anti-mPC antibody (R&D Systems) was immobilized was blocked with 5% casein, washed with phosphate buffer, and diluted samples were added. After 2 hr, the plate was washed with phosphate buffer, and then HRP-labeled anti-mPC antibody (GENTEX) was added and incubated for another hour. After washing with phosphate buffer, a peroxidase substrate (KPL Protein Research Product) was added, and the absorbance at 415 nm was measured.
Coagulation time [activated partial thromboplastin time (APTT)]: As in Example 3, measured using a CS510 (Sysmex).
Factor V activity: Measured using a CS1600 (Sysmex) by the one-stage clotting assay.
Factor VIII activity: Measured using CS1600 (Sysmex) by one-stage clotting assay.
AAV genome amount: SV40 polyadenylation signal contained in the vector was evaluated by quantitative PCR. The primer and probe sequences used are as follows: 5′-AGCAATAGCATCACAAATTTCACAA-3′ (sense) (SEQ ID NO: 19) 5′-CCAGACATGATAAGATACATTGATGAGTT-3′ (antisense) (SEQ ID NO: 20) 5′-AGCATTTTTTTTCACTGCATTCTAGTTGTGGTTTTGTC-3′ (FAM probe) (SEQ ID NO: 21)
Pathological thrombus formation dependent on reactive oxygen: Under anesthesia, to the mice were intravenously administered anti-platelet GPIbp antibody (DyLight488 conjugated) (Emfret Analytics GmbH & Co.), rhodamine B (Sigma Aldrich)- or Texas Red-conjugated dextran (Thermo Fisher Scientific), and Hoechst 33342 (Thermo Fisher Scientific) under anesthesia. In addition, hematoporphyrin (Sigma Aldrich) was administered to prevent vascular wall damage caused by reactive oxygen species. Laser-induced thrombus formation in the testicular vein was then observed using a confocal microscope (Leica TCS SP8).

### Example 7

To newborn mice born from the mating of heterozygotes of protein C-deficient mice produced by genome editing targeting exon 9 were administered two types of AAV vectors (vector expressing SaCas9 and vector carrying mPC variant) to express activated PC in the newborn liver by genome editing. Similar to the mating of hemophilia A mice (F8−/−) (provided by Dr. Kazazian, University of Pennsylvania, available from Jackson), expression of activated protein C by genome editing resulted in survival of protein C-deficient mice (FIG. 9A). Mouse protein C antigen amount (FIG. 9B), factor V activity (FIG. 9C), factor VIII activity (FIG. 9D), and coagulation time [activated partial thromboplastin time (APTT)](FIG. 9E) were measured in the same manner as in Example 6.

### INDUSTRIAL APPLICABILITY

By producing the polypeptide or partial polypeptide thereof, protein or partial protein thereof, nucleic acid, vector, host cell, or host cell population of the present invention, it becomes possible to 1) produce activated protein C as a recombinant preparation, and 2) link same to effective gene therapy for protein C deficiency. By producing activated protein C by recombinant means, a safe protein preparation without the risk of infectious diseases can be obtained. The blood molar concentration of protein C is at the same level as that of factor IX, which is deficient in hemophilia B, and considering the results of human clinical trials to date, a sufficiently therapeutic level of blood molar concentration of protein C can be obtained by the administration of an AAV vector.

This application is based on a patent application No. 2022-035659 filed in Japan (filing date: Mar. 8, 2022), the contents of which are incorporated in full herein.

1. A polypeptide or a partial polypeptide thereof,
comprising an amino acid sequence represented by
formula:
A₁—A₂—A₃ (I)
wherein A₁ is an amino acid sequence comprising an amino acid sequence of a light chain of protein C or a homologue thereof, A₂ is an amino acid sequence constituting a self-cleaving site, a A₃ is an amino acid sequence comprising an amino acid sequence of a heavy chain of protein C or a homologue thereof, and the C-terminal amino acid of A₁ and the N-terminal amino acid of A₃ are Arg at position 211 and Leu at position 212 of human protein C, respectively,
wherein a dimeric protein consisting of fragments on the N-terminal side and C-terminal side of the cleaving site of A₂, or a partial protein thereof, has protein C activity,
wherein the polypeptide satisfies the following conditions:
(1) an amino acid sequence (formula: A₁—A₃ (II)) in which A₁ (N-terminal side) and A₃ (C-terminal side) are linked comprises the amino acid sequence of SEQ ID NO: 2,
(2) an amino acid sequence in which A₁ (N-terminal side) and A₃ (C-terminal side) are linked comprises an amino acid sequence in which 1 to 45 amino acids have been deleted, substituted, inserted, or added in the amino acid sequence represented by SEQ ID NO: 2, or
(3) an amino acid sequence in which A₁ (N-terminal side) and A₃ (C-terminal side) are linked comprises an amino acid sequence having 90% or more identity to the amino acid sequence represented by SEQ ID NO: 2, and
A₂ is selected from the group consisting of RKRRKR (SEQ ID NO: 3), KRRKR (SEQ ID NO: 4), RKRRKRRKR (SEQ ID NO: 8), and RKRRKRRKRRKR (SEQ ID NO: 9).

2. (canceled)

3. (canceled)

4. The polypeptide or partial polypeptide thereof according to claim 1, wherein A₂ is RKRRKR (SEQ ID NO: 3) or KRRKR (SEQ ID NO: 4).

5. The polypeptide or partial polypeptide thereof according to claim 1, wherein the polypeptide comprises the amino acid sequence represented by SEQ ID NO: 13 or SEQ ID NO: 14.

6. A dimeric protein or a partial protein thereof consisting of fragments on the N-terminal side and C-terminal side of the A₂ cleaving site of the polypeptide or partial polypeptide according to claim 1, wherein the protein or partial protein thereof has protein C activity.

7. A nucleic acid comprising a nucleotide sequence encoding the polypeptide or partial polypeptide thereof according to claim 1.

8. A vector comprising the nucleic acid according to claim 7.

9. The vector according to claim 8, which is an expression vector.

10. The vector according to claim 8, which is a donor vector.

11. The vector according to claim 8, which is a plasmid vector.

12. The vector according to claim 8, which is a viral vector.

13. The vector according to claim 12, which is an adeno-associated virus (AAV) vector.

14. A host cell comprising the vector according to claim 8.

15. A host cell population comprising the host cell according to claim 14.

16. A pharmaceutical composition comprising the polypeptide or partial polypeptide thereof according to claim 1.

17. A pharmaceutical composition comprising the vector according to claim 10 and a vector comprising a nucleic acid encoding a nucleic acid metabolic enzyme.

18. The pharmaceutical composition according to claim 17, wherein the nucleic acid metabolic enzyme is a CRISPR/Cas9 system nucleic acid metabolic enzyme, and the composition further comprises a vector comprising a nucleic acid encoding a guide RNA, or comprises a vector comprising a nucleic acid encoding a guide RNA together with a nucleic acid encoding a nucleic acid metabolic enzyme.

19. The pharmaceutical composition according to claim 16, which is for suppressing blood coagulation.

20. The pharmaceutical composition according to claim 16, which is for treating or preventing thrombosis.

21. The pharmaceutical composition according to claim 20, wherein the thrombosis is selected from the group consisting of venous thrombosis, disseminated intravascular coagulation, (neonatal) purpura fulminans, deep vein thrombosis pulmonary thromboembolism, and thrombosis associated with new coronavirus infection.

22. A method for producing a protein C-expressing cell, comprising introducing the vector according to claim 8 into a mammalian cell in vitro.

23. A method for producing a recombinant preparation of protein C, comprising producing a protein C-expressing cell by the method according to claim 22, and isolating, purifying, and formulating activated protein C from the cell.


SEQUENCE LISTING (23 sequences)

SEQ ID NO: 1 - DNA, 1386 bp, Homo sapiens
  source 1..1386: mol_type = other DNA
atgtggcagctcacaagcctcctgctgttcgtggccacctggggaatttccggcacaccagctcctcttgactcagtgttctccagcagcgagcgtgcccaccaggtgctgcggatccgcaaacgtgccaactccttcctggaggagctccgtcacagcagcctggagcgggagtgcatagaggagatctgtgacttcgaggaggccaaggaaattttccaaaatgtggatgacacactggccttctggtccaagcacgtcgacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcgaggtgagcttcctcaattgctctctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtggtcctgctggactcaaagaagaagctggcctgcggggcagtgctcatccacccctcctgggtgctgacagcggcccactgcatggatgagtccaagaagctccttgtcaggcttggagagtatgacctgcggcgctgggagaagtgggagctggacctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttag

SEQ ID NO: 2 - protein, 419 aa, Homo sapiens
ANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 3 - protein, 6 aa, synthetic construct
  REGION 1..6: note = Synthetic peptide
RKRRKR

SEQ ID NO: 4 - protein, 5 aa, synthetic construct
  REGION 1..5: note = Synthetic peptide
KRRKR

SEQ ID NO: 5 - protein, 4 aa, synthetic construct
  REGION 1..4: note = Synthetic peptide
RHQR

SEQ ID NO: 6 - protein, 4 aa, synthetic construct
  REGION 1..4: note = Synthetic peptide
RSKR

SEQ ID NO: 7 - protein, 19 aa, synthetic construct
  REGION 1..19: note = Synthetic peptide
ATNFSLLKQAGDVEENPGP

SEQ ID NO: 8 - protein, 9 aa, synthetic construct
  REGION 1..9: note = Synthetic peptide
RKRRKRRKR

SEQ ID NO: 9 - protein, 12 aa, synthetic construct
  REGION 1..12: note = Synthetic peptide
RKRRKRRKRRKR

SEQ ID NO: 10 - protein, 18 aa, synthetic construct
  REGION 1..18: note = Synthetic peptide
EGRGSLLTCGDVEENPGP

SEQ ID NO: 11 - protein, 20 aa, synthetic construct
  REGION 1..20: note = Synthetic peptide
QCTNYALLKLAGDVESNPGP

SEQ ID NO: 12 - protein, 22 aa, synthetic construct
  REGION 1..22: note = Synthetic peptide
VKQTLNFDLLKLAGDVESNPGP

SEQ ID NO: 13 - protein, 425 aa, synthetic construct
  REGION 1..425: note = modified protein C
ANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRRKRRKRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 14 - protein, 424 aa, synthetic construct
  REGION 1..424: note = modified protein C
ANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRKRRKRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 15 - protein, 169 aa, Homo sapiens
ANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPR

SEQ ID NO: 16 - protein, 250 aa, Homo sapiens
LIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 17 - DNA, 1404 bp, synthetic construct
  misc_feature 1..1404: note = modified protein C DNA
  source 1..1404: mol_type = other DNA
atgtggcagctcacaagcctcctgctgttcgtggccacctggggaatttccggcacaccagctcctcttgactcagtgttctccagcagcgagcgtgcccaccaggtgctgcggatccgcaaacgtgccaactccttcctggaggagctccgtcacagcagcctggagcgggagtgcatagaggagatctgtgacttcgaggaggccaaggaaattttccaaaatgtggatgacacactggccttctggtccaagcacgtcgacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcgaggtgagcttcctcaattgctcgctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgagacacagaagaccaagaagaccaagtagatccgcggaggaagcggagaaagcggctcattgatgggaagatgaccaggcggggagacagcccctggcaggtggtcctgctggactcaaagaagaagctggcctgcggggcagtgctcatccacccctcctgggtgctgacagcggcccactgcatggatgagtccaagaagctccttgtcaggcttggagagtatgacctgcggcgctgggagaagtgggagctggacctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttag

SEQ ID NO: 18 - DNA, 1404 bp, synthetic construct
  misc_feature 1..1404: note = modified protein C DNA
  source 1..1404: mol_type = other DNA
atgtggcagctgacttcactgctgctgtttgtcgctacttggggaattagtggaactcctgctcctctggactctgtcttctcctctagcgagagagcccaccaggtgctgaggatccgcaagcgggccaactccttcctggaggagctgagacacagctccctggagagggagtgcatcgaggagatctgtgacttcgaggaggccaaggagatctttcagaatgtggacgataccctggccttttggtccaagcacgtggacggcgatcagtgcctggtgctgccactggagcacccctgtgcctctctgtgctgtggccacggcacatgcatcgacggcatcggctccttctcttgcgattgtaggtccggatgggagggccgcttctgccagagagaggtgtcttttctgaactgtagcctggataatggcggatgcacccactactgtctggaggaagtgggatggcggagatgctcctgtgcaccaggctataagctgggcgacgatctgctgcagtgccacccagccgtgaagtttccttgtggcagaccatggaagaggatggagaagaagcgcagccacctgaagcgggacaccgaggatcaggaggaccaggtggatcctcgcaggaagcggagaaagcggctgatcgacggcaagatgacaagacgcggcgatagcccatggcaggtggtgctgctggacagcaagaagaagctggcatgtggagccgtgctgatccacccatcctgggtgctgacagccgcccactgtatggacgagtctaagaagctgctggtgcggctgggcgagtacgatctgcggagatgggagaagtgggagctggacctggatatcaaggaggtgttcgtgcaccccaactatagcaagtccaccacagacaatgatatcgccctgctgcacctggcacagcctgccaccctgtctcagacaatcgtgcctatctgtctgcctgactctggcctggcagaaagagagctgaaccaggcaggacaggagacactggtgacaggctggggctaccactctagccgggagaaggaggccaagagaaaccggaccttcgtgctgaacttcatcaagatccccgtggtgcctcacaatgagtgctctgaagtgatgagcaacatggtgtccgagaatatgctgtgcgccggcatcctgggcgacagacaggatgcatgtgaaggcgattccggcggccctatggtggcatctttccacggcacctggtttctggtgggcctggtgtcttggggcgagggatgtggcctgctgcacaactacggcgtgtatacaaaggtgagcaggtatctggactggattcacgggcacattagagacaaagaagcacctcagaagagttgggcaccataa

SEQ ID NO: 19 - DNA, 25 bp, synthetic construct
  source 1..25: PCR_primers = fwd_seq: agcaatagcatcacaaatttcacaa, rev_seq: ccagacatgataagatacattgatgagtt; mol_type = other DNA
agcaatagcatcacaaatttcacaa

SEQ ID NO: 20 - DNA, 29 bp, synthetic construct
  source 1..29: PCR_primers = fwd_seq: agcaatagcatcacaaatttcacaa, rev_seq: ccagacatgataagatacattgatgagtt; mol_type = other DNA
ccagacatgataagatacattgatgagtt

SEQ ID NO: 21 - DNA, 36 bp, synthetic construct
  source 1..36: mol_type = other DNA; note = FAM probe
agcatttttttcactgcattctagttgtggtttgtc

SEQ ID NO: 22 - protein, 10 aa, synthetic construct
QVDPRLIDGK

SEQ ID NO: 23 - protein, 7 aa, synthetic construct
QGNSEDY